(19) United States
(12) Patent Application Publication
(10) Pub. No.: US 2006/0171919 A1
(43) Pub. Date: Aug. 3, 2006

(54) Targeted polypeptides

(75) Inventors: Michael G. Rosenblum, Sugar Land, TX (US); Lawrence Cheung, Houston, TX (US); Mi-Ae Lyu, Houston, TX (US)

Correspondence Address: Fulbright & Jaworski L.L.P.;Fulbright Tower, Suite 5100, 1301 McKinney, Houston, TX, 77010-3095, US

(73) Assignee: Research Development Foundation, Carson City, NV (US)

(21) Appl. No.: 11/345,661

(22) Filed: Feb. 1, 2006

Related U.S. Application Data
(60) Provisional application No. 60/649,478, filed on Feb. 1, 2005.

(51) Int. Cl. A61K 38/19; C07K 14/525; C07K 14/415; C07K 14/195; A61K 36/47

(52) U.S. Cl. 424/85.1; 530/351; 424/234.1; 424/731

(57) ABSTRACT

The present invention regards targeted BLyS polypeptides capable of binding to BLyS receptors and that deliver a cytotoxic agent, such as a cytotoxic peptide, for example. In particular aspects, the cytotoxic agent comprises at least part of rGelonin. These compositions are useful for treating, preventing, and/or monitoring therapy for a B-cell proliferative disorder.

The present invention claims priority to U.S. Provisional Application Ser. No. 60/649,478, filed Feb. 1, 2005, which is incorporated by reference herein in its entirety.

### FIELD OF THE INVENTION

The present invention is directed at least to the fields of cell biology, molecular biology, cancer biology, and medicine. More particularly, the present invention relates to compositions comprising a BLyS polypeptide conjugated to a cytotoxic agent, and the use of such compositions in therapy.

### BACKGROUND OF THE INVENTION

B lymphocyte stimulator is a member of the tumor necrosis factor (“TNF”) superfamily that induces both in vivo and in vitro B-cell proliferation and differentiation (Moore et al., Science 285: 260-263 (1999)). BLyS is distinguishable from other B-cell growth and differentiation factors such as IL-2, IL-4, IL-5, IL-6, IL-7, IL-13, IL-15, CD40L, or CD27L (CD70) by its monocyte-specific gene and protein expression pattern and its specific receptor distribution and biological activity on B lymphocytes. BLyS expression is not detected on natural killer (“NK”) cells, T cells or B-cells, but is restricted to cells of myeloid origin. BLyS expression on resting monocytes is upregulated by interferon-gamma (IFN-gamma). The gene encoding BLyS has been mapped to chromosome 13q34.

Human BLyS is expressed as a 285 amino acid type II membrane-bound polypeptide and a soluble 152 amino acid polypeptide (Moore et al., 1999 supra). The membrane-bound form of BLyS has a predicted transmembrane spanning domain between amino acid residues 47 and 73. The NH₂-terminus of the soluble form of BLyS begins at Ala¹³⁴ of the membrane-bound form of BLyS. Soluble recombinant BLyS has been shown to induce in vitro proliferation of murine splenic B-cells and to bind to a cell-surface receptor on these cells (Moore et al., 1999 supra). Soluble BLyS administration to mice has been shown to result in an increase in the proportion of CD45R^{dull}, Ly6D^{bright} (also known as ThB) B-cells and an increase in serum IgM and IgA levels (Moore et al., 1999 supra). Thus, BLyS displays a B-cell tropism in both its receptor distribution and biological activity.

Successful development of tumor-targeted therapeutic agents is dependent, in part, on the site-specific delivery of therapeutic agents and also on the biological activity of the delivered agent. Monoclonal antibodies have been employed to impart selectivity to otherwise indiscriminately cytotoxic agents such as toxins, radioneuclides, and growth factors (Williams et al., 1990; Rowlinson-Busza et al., 1992; Wahl, 1994). One such molecule is gelonin, a 29-kDa ribosome-inactivating plant toxin with a potency and mechanism of action similar to ricin A-chain (RTA) but with improved stability and reduced toxicity (Stirpe et al., 1992; Rosenblum et al., 1995). Previous studies have identified and examined the biological properties of numerous chemical conjugates of the plant toxin gelonin and various antibodies (Boyle et al., 1995; Xu et al., 1996; Rosenblum et al., 1999). In previous studies, antibody ZME-018 was chemically coupled to purified gelonin, and this immunoconjugate demonstrated specific cytotoxicity against antigen-positive melanoma cells both in tissue culture and in human tumor xenograft models (Rosenblum et al., 1991; Mujoo et al., 1995).

There exists a need for improved therapeutic agents with specificity for abnormally proliferating B-cells.

### SUMMARY OF THE INVENTION

The present invention concerns methods of generating and using molecules that possess targeting activity, such as to a cancer cell, including in a tumor, for example, and cytotoxic activity. The molecule may comprise a conjugated polypeptide, for example. The present invention also includes compositions that are generated from these conjugated polypeptides. Proteinaceous conjugates of the invention, for example, include a compound that comprises both a toxin and a BLyS polypeptide. In some embodiments, the conjugated polypeptides are engineered recombinantly to produce a fusion protein. Conjugated compounds may be also attached to one another by a linker, for example.

In specific embodiments of the invention, the targeting activity and the cytotoxic activity are provided to the molecule by a separate moiety of the molecule, although alternatively one moiety may comprise part or all of both targeting and cytotoxic activities.

One with skill in the art realizes that “BLyS” may be interchangeable with BAFF, BLYS, TALL1, THANK, ZTNF4, TALL-1, TNFSF20, and delta BAFF polynucleotides or polypeptides, although in alternative embodiments it is not interchangeable. One of skill in the art recognizes how to determine if the molecule that is potentially interchangeable with BLyS would be suitable, such as by disclosure provided herein and/or available in the art. As envisioned by the present invention, SEQ ID NO:1 refers to the full-length 285 amino acid human BLyS polypetide. SEQ ID NO:20 refers to the polynucleotide sequence of human BLyS. Amino acid residues 134-285 of SEQ ID NO:1 comprise a soluble isoform of the BLyS polypeptide, which in certain embodiments is the isoform that is used in the conjugated polypeptides of the present invention. Mouse (polypeptide SEQ ID NO:2; nucleotide SEQ ID NO:3) BLyS, or any other orthologs of BLyS are also contemplated by the present invention. Functional equivalents of BLyS are also appropriate for use with the targeted conjugated polypeptides of the present invention. For example, polypeptides retaining BLyS activity with at least 80% sequence homology, at least 85% sequence homology, and at least 90% sequence homology with any of SEQ ID NO:1 or SEQ ID NO:2 are contemplated. In a further specific embodiment, the amino acid sequence of BLyS is at least about 40, at least about 50, at least about 75, at least about 100, at least about 125, at least about 150, at least about 175, at least about 200, at least about 225, at least about 250, or at least about 275 contiguous amino acids from SEQ ID NO:1 or SEQ ID NO:2. In other embodiments, functional equivalents comprise at least the D-E loop involved in receptor recognition. (See Oren et al., Nat Struct Biol. 2002 April; 9(4):288-92).

In some embodiments wildtype BLyS may be utilized, and in other embodiments mutant BLyS may be utilized. Exemplary BLyS mutants include a mutation at Cys146 (Chen et al., 2002; 2004; 2005), and in specific embodiments the mutation is to alanine or valine. BLyS mutants employed in the invention will retain the ability to target B cells, such as by retaining the ability to bind at least one BLyS receptor. A BLyS mutant may enhance any activity over a wild type BLyS, including the ability to bind a B cell, such as through a BLyS receptor.

The BLyS targeted polypeptides of the present invention are targeted to cells that express a BLyS receptor, such as TNFRSF13B/TACI (SEQ ID NO:4), TNFRSF17/BCMA (SEQ ID NO: 5), and TNFRSF13C/BAFFR (SEQ ID NO: 6). In certain embodiments of the invention, the conjugated polypeptides of the present invention will specifically bind a cell expressing a functional equivalent of SEQ ID NO:4, SEQ ID NO:5, or SEQ ID NO:6. In certain embodiments of the invention, it is contemplated that cells expressing a BLyS receptor may be associated with abnormal B-cell proliferation. One with skill in the art realizes that BLyS may form dimers or trimers in order to mediate receptor recognition. It will be understood that proteinaceous compositions of conjugated polypeptides as envisioned herein will allow for the formation of BLyS dimers, trimers, or any multimeric protein complex.

In certain embodiments, the BLyS polypeptide is attached to a molecule. In preferred embodiments, the attachment is a covalent attachment. In additional embodiments, the molecule is a cytotoxic agent, drug, a chemotherapeutic agent, a radioisotope, a pro-apoptosis agent, an anti-angiogenic agent, a hormone, a cytokine, a growth factor, a peptide, a protein, an antibiotic, an enzyme (such as Granzyme B or Granzyme A, for example), an antibody, a Fab fragment of an antibody, an imaging agent, a nucleic acid or an antigen, for example. These molecules are representative only. Molecules within the scope of the present invention include virtually any molecule that may be attached to a BLyS polypeptide and administered to a human subject. In preferred embodiments, the pro-aptoptosis agent is gramicidin, magainin, mellitin, defensin, or cecropin, for example. In other preferred embodiments, the anti-angiogenic agent is thrombospondin, angiostatin, endostatin or pigment epithelium-drived factor. In further preferred embodiments, the cytokine is interleukin 1 (IL-1), IL-2, IL-5, IL-10, IL-11, IL-12, IL-18, interferon-γ (IF-γ), IF-α, IF-β, tumor necrosis factor-α (TNF-α), or GM-CSF (granulocyte macrophage colony stimulating factor), for example. Such examples are representative only and are not intended to exclude other pro-apoptosis agents, anti-angiogenic agents or cytokines known in the art.

In some embodiments of the invention, a recombinant gelonin peptide toxin is provided (shown in SEQ ID NO:7), which is disclosed in U.S. Pat. No. 5,631,348, and which is herein incorporated by reference. The recombinant gelonin toxin or the present invention may be any portion or fragment of SEQ ID NO:7 that retains toxin activity. In certain embodiments, the gelonin comprises residues 110-210 of SEQ ID NO:7. In a further specific embodiment, the amino acid sequence of rGelonin is at least about 30, at least about 40, at least about 50, at least about 75, at least about 100, at least about 125, at least about 150, at least about 175, at least about 200, at least about 225, or at least about 250 contiguous amino acids from SEQ ID NO:7. Other compounds of the present invention include a recombinant gelonin toxin that contains the core toxin region in addition to having at least 5, 10, 20, 30, 40, 50, 60, 70, 80, 90, 100 or more contiguous amino acid residues of SEQ ID NO:7 in addition to the core toxin region. In other embodiments of the invention, gelonin comprises SEQ ID NO:21 (Genbank Accession No. P33186). In some embodiments wildtype rGel may be utilized, and in other embodiments mutant rGel may be utilized. In specific embodiments of the invention, an rGel molecule in accordance with U.S. patent application Ser. No. 10/074,596, filed Feb. 12, 2002 and which is incorporated by reference herein in its entirety, is used in the invention.

In one embodiment of the invention, an exemplary conjugated polypeptide is provided having the sequence shown in SEQ ID NO: 8. An exemplary polynucleotide encoding the conjugated polypeptide is shown in SEQ ID NO: 9. In another embodiment of the invention, an exemplary conjugated polypeptide is provided having the sequence shown in SEQ ID NO:10. A polynucleotide encoding the conjugated polypeptide is shown in SEQ ID NO:11.

In other embodiments of the invention, the cytotoxic agent provided by the present invention may be abrin, dodecandrin, tricosanthin, tricokirin, bryodin, mirabilis antiviral protein, barley ribosome-inactivating protein (BRIP), pokeweed antiviral proteins (PAPs), saporins, luffins, and/or momordins, for example.

Polypeptides of the present invention may be conjugated, in certain aspects of the invention. Generally, these conjugated polypeptides are at least five amino acids in length. In certain embodiments of the invention, conjugated polypeptides are about 5-about 10 amino acids in length, about 5-about 50 amino acids in length, about 5-about 100 amino acids in length, about 5-about 150, about 5-about 500, about 5-about 1000, or about 5-about 5000 amino acids in length, for example.

The conjugation of the polypeptides of the present invention may be produced by any suitable means including, for example, by both chemical conjugation and “genetic conjugation,” such as recombinant fusion proteins comprising the BLyS polypeptide operatively linked to a cytotoxic peptide. The contemplated conjugation with the BLyS polypeptide includes conjugation at the N-terminal region of the protein (within the first 100 amino acids), internal region (between the N-terminal and C-terminal regions), and/or C-terminal region of the protein (within the last 100 amino acids). Conjugation techniques envisioned for use in the present invention are described in detail herein.

Conjugated polypeptides of the invention can be exogenously expressed. In specific embodiments of the invention, a polypeptide comprises a fusion or chimeric polypeptide. A chimeric polypeptide comprises all or a discrete part of two or more polypeptides. A discrete part of a polypeptide refers to an amino acid region that contains an identifiable function or activity. A fusion protein is a type of chimeric protein in which a first polypeptide or part of the first polypeptide is linked end-to-end to a second polypeptide or a part of the second polypeptide.

The present invention also provides methods for treating a subject with a B-cell proliferative disorder comprising administering to a subject with the disorder a therapeutically effective amount of one or more molecules of the invention, including conjugated polypeptides. The term “B-cell-proliferative disorder” denotes malignant as well as non-malignant cell populations, which often appear to differ from the surrounding tissue both morphologically and genotypically. The cytotoxic polypeptide may be linked to the BLyS polypeptide through a variety of means known to one with skill in the art and described in further detail herein.

Exemplary B-cell proliferative disorders that may be treated by present invention include at least the following: B-cell chronic Lymphocytic leukemia/small lymphocytic lymphoma B-cell prolymphocytic leukemia, Immunocytoma/lymphoplasmacytic lymphoma (+/−Waldenstrom's macroglobulinemia), Mantle cell lymphoma, Marginal Zone B-cell Lymphoma of mucosa-associated lymphoid tissue (MALT) type, Splenic marginal zone B-cell Lymphoma (+/−villous Lymphocytes), Hairy cell leukemia, Diffuse large B-cell Lymphoma, Mediastinal (Thymic) large B-cell Lymphoma, Intravascular large B-cell Lymphoma, Burkitt Lymphoma, Plasma cell myeloma (multiple myeloma), Monoclonal gammopathy of undetermined significance (MGUS), Indolent myeloma, Smoldering myeloma, Osteosclerotic myeloma (POEMS syndrome), Plasma cell leukemia, Non-secretory myeloma, Plasmacytomas, Solitary plasmacytoma of bone, Extramedullary plasmacytoma, Waldenstrom's macroglobulinemia, Heavy Chain Disease (HCD), Immunoglobulin deposition diseases, Systemic light chain disease, and Primary amyloidosis.

In particular aspects of the invention, the compositions and methods are directed to B-cell proliferative disorders that are refractory to a treatment. The disorder may be initially refractory to the treatment, or the disorder may become refractory to the treatment during an initial or subsequent treatment.

Other embodiments of the invention concern compositions of the present invention for the prevention and/or treatment of at least one symptom of a B-cell proliferative disorder. In particular embodiments of the invention, compositions of the present invention are employed for the prevention and/or treatment of an auto-immune disorder, such as arthritis or lupus, for example. Concerning preventive embodiments, the symptom may be completely prevented from occurring or it may be delayed in manifesting. Concerning treatment embodiments, the symptom may be completely eliminated or may be partially improved.

In other aspects of the invention, the methods and compositions are utilized for an individual that has received another treatment for the same B-cell proliferative disorder, that will receive another treatment, or that is receiving the treatment. Any additional therapy may be employed, although in particular embodiments the additional therapy comprises radiation, chemotherapy, surgery, gene therapy, immunotherapy, hormone therapy, or a combination thereof.

In specific embodiments, there is a method of treating an individual with a B-cell proliferative disorder comprising administering to the patient a therapeutically effective amount of a composition, such as a conjugated polypeptide, comprising a BLyS polypeptide conjugatged to a cytotoxic peptide, in particular aspects the method further comprises administering to the individual an agent that increases the expression of the BLyS receptor.

In additional embodiments of the invention, a BLyS receptor may be introduced into a host cell and expressed in the host cell, allowing BLyS to target cells outside of its natural tropism. In certain embodiments, a polynucleotide comprising nucleic acid sequence that encodes a BLyS conjugate (such as a fusion protein) of the invention is regulated in expression by a tissue-specific promoter. For example, a BLyS receptor molecule, such as SEQ ID NO:4 (gene sequence SEQ ID NO:12), is introduced by a suitable vector into a host liver cell. The expression of the BLyS receptor is controlled by a hepatoma-specific promoter. Thus, BLyS conjugated polypeptides may be targeted to hepatoma cells for therapy. In other embodiments of the invention, the conjugated polypeptides may be linked to an imaging agent in order to track the progress of polypeptide targeting in a patient.

In an additional embodiment of the invention, there is a method of selectively targeting a cell expressing a BLyS receptor comprising contacting the cell with a conjugated polypeptide comprising a BLyS polypeptide conjugated to a cytotoxic peptide.

In another embodiment of the invention, there is a method of monitoring therapy for B-cell proliferative disorder comprising administering to the patient a therapeutically effective amount of a conjugated polypeptide comprising a BLyS polypeptide conjugated to a cytotoxic peptide and an imaging agent.

The present invention concerns multipolypeptide compositions in which more than one polypeptide entity is presented as a single compound. Thus, a BLyS protein may be attached to a gelonin toxin, for example, and to second, third, fourth, fifth, sixth or more polypeptides.

In other embodiments of the invention, there are kits for the treatment and/or prevention of B-cell proliferative disorders comprising a composition including BLyS polypeptide conjugated to another molecule, such as a cytotoxic agent or pharmaceutical agent, for example. In specific embodiments, the composition comprises a fusion protein of rGel and BLyS, and/or a polynucleotide encoding same.

Thus, in embodiments of the invention there is a composition comprising a BLyS polypeptide conjugated to an additional molecule, including a molecule that is non-identical to the BLyS polypeptide. In specific aspects, the additional molecule is not a homolog of BLyS. In particular aspects of the invention, the additional molecule comprises a pharmaceutical agent, a chelate, or a cytotoxic agent, for example. The components of the composition may be comprised as a fusion protein or may be chemically conjugated, for example. The composition of the invention may comprise a recombinant polypeptide and/or the polynucleotide encoding the recombinant polypeptide. In specific aspects, the composition further comprises a radioactive agent, a cell imaging agent, or both. The composition may be comprised in a pharmaceutically acceptable carrier.

In specific aspects of the BLyS polypeptide, the polypeptide comprises a B-cell targeting domain, comprises a D-E receptor recognition loop, comprises all or a functional portion of SEQ ID NO:1 or SEQ ID NO:2, and/or comprises a functional equivalent of BLyS, wherein said functional equivalent possesses at least 80% sequence homology to SEQ ID NO:1 or SEQ ID NO:2.

In embodiments wherein a cytotoxic agent is employed, the cytotoxic agent may be of any suitable kind, although in particular embodiments, the cytotoxic agent comprises a peptide, a polypeptide, or a small molecule, for example. In specific aspects, the cytotoxic peptide comprises a gelonin peptide, which in exemplary embodiments is 5′ to the BLyS polypeptide. In specific embodiments of the invention, the gelonin peptide comprises SEQ ID NO:7. In further specific embodiments, the gelonin peptide comprises amino acid residues 110-210 of SEQ ID NO:7.

In specific embodiments of the invention, the cytotoxic agent is selected from the group consisting of ricin A, diphtheria toxin, abrin, dodecandrin, tricosanthin, tricokirin, bryodin, mirabilis antiviral protein, barley ribosome-inactivating protein (BRIP), pokeweed antiviral protein (PAPs), saporin, luffin, Pseudomonas exotoxin, and momordin, for example.

In certain aspects of the invention, the composition comprises SEQ ID NO:8 and/or SEQ ID NO:10, for example.

In particular aspects of the invention, there is a host cell comprising a composition of the invention. In other particular aspects of the invention, there is an isolated polynucleotide encoding at least part of a composition of the invention, including all of the composition. In specific embodiments, the polynucleotide comprises SEQ ID NO:9 or SEQ ID NO:11.

In additional embodiments of the invention, there is a method of treating an individual with a B-cell proliferative disorder comprising administering to the patient a therapeutically effective amount of a composition comprising a BLyS polypeptide conjugated to an additional molecule, such as a cytotoxic agent, for example. The method may further comprise administering to the individual an agent that increases the expression of the BLyS receptor, for example. In specific aspects of the invention, the BLyS receptor is selected from the group consisting of TNFRSF13B/TACI (SEQ ID NO:4), TNFRSF17/BCMA (SEQ ID NO: 5), and TNFRSF13C/BAFFR (SEQ ID NO: 6).

In an embodiment of the invention, there is a method of selectively targeting a cell expressing a BLyS receptor comprising contacting the cell with a composition comprising a BLyS polypeptide conjugated to a cytotoxic agent.

In an additional embodiment of the invention, there is a method of monitoring therapy in an individual with B-cell proliferative disorder, comprising administering to the individual a therapeutically effective amount of a composition comprising a BLyS polypeptide conjugated to a cytotoxic agent and an imaging agent.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth herein. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DESCRIPTION OF THE DRAWINGS

For a more complete understanding of the present invention, reference is now made to the following descriptions taken in conjunction with the accompanying drawings.

FIG. 1 shows an exemplary orientation of BLyS and rGel.

FIG. 2 shows exemplary DNA (SEQ ID NO:9) and protein sequence (SEQ ID NO:8) of a BLyS/rGel fusion toxin.

FIG. 3 shows exemplary DNA (SEQ ID NO:11) and protein sequence (SEQ ID NO:10) of a rGel/BLyS fusion toxin.

FIG. 4 illustrates construction of an exemplary fusion toxin rGel/BLyS. A fusion toxin rGel/BLyS was generated containing rGel at the N-terminus followed by a G4S peptide tether to the BLyS molecule using a splice overlap extension PCR method. The recombinant rGel/BLyS DNA construct was introduced into Kpn I and Xho I restriction enzyme site of pET-32a vector to construct the expression vector pET32rGel/BLyS.

FIG. 5 shows the purification of rGel/BLyS fusion toxin. As shown in the Coomassie-stained SDS-PAGE analysis of the rGel/BLyS fusion toxin, the Mᵣ of rGel/BLyS was 45 kDa, demonstrating a 1:1 molar ratio of BLyS and rGel (left panel). Western blot analysis using anti-gelonin antibody or anti-BlyS antibody demonstrated that the rGel/BLyS fusion toxin contained toxin and BLyS component in the fusion toxin (right panel).

FIG. 6 shows cell-free protein synthesis inhibitory activity of the rGel/BLyS fusion toxin. To examine the n-glycosidic activity of the rGel component of the rGel/BLyS fusion toxin, this material was added to an in vitro protein translation assay using [³H]-leucine incorporation by isolated rabbit reticulocytes. Inhibition curves for the rGel/BLyS fusion toxin and native rGel were compared.

FIG. 7 is a comparison of the cytotoxic activity of rGel/BLyS and BLyS/rGel fusion toxin against JEKO mantle cell line. JEKO mantle cell lines were seeded (5×10³/well) in flat-bottom 96-well microtiter plates and rGel, rGel/BLyS, or BLyS/rGel were added in quadruplicate wells. After 96 hr, 75 μl of XTT labeling mixture was added to each well, after which the cells incubated for another 4 hr. The spectrophotometric absorbance was measured at 450 nm using an ELISA reader.

FIGS. 8A-8M show dose-response curves of rGel/BLyS fusion toxin against various tumor cell lines. Jurkat (8A), KBM-5 (8B), THP-1 (8C), HL-60 (8D), IM-9 (8E), MM1.S (8F), MM1.R (8G), RPMI8226 (8H), 8226/LR-5 (8I), JEKO (8J), SP53 (8K), Mino (8L), and Granta (8M). Thirteen tumor cell lines were seeded (5×10³/well) in flat-bottom 96-well microtiter plates and rGel, or rGel/BLyS were added in quadruplicate wells. After 96 hr, 75 μl of XTT labeling mixture was added to each well, after which the cells incubated for another 4 hr. The spectrophotometric absorbance was measured at 450 nm using an ELISA reader.

FIG. 9 shows the specificity of rGel/BLyS fusion toxin against BLyS receptor expressing JEKO mantle cell lines. JEKO mantle cell lines were seeded (5×10³/well) in flat-bottom 96-well microtiter plates and BLyS, rGel, CTP/rGel, and rGel/BLyS were added in quadruplicate wells. After 96 hr, 75 μl of XTT labeling mixture was added to each well, after which the cells incubated for another 4 hr. The spectrophotometric absorbance was measured at 450 nm using an ELISA reader.

FIG. 10 provides dose-response curves of rGel/BLyS fusion toxin against Dexamethasone-sensitive (MM1.S) and -resistant (MM1.R) multiple myeloma cell lines. MM1.S and MM1.R cell lines were seeded (5×10³/well) in flat-bottom 96-well microtiter plates and rGel, Dex, or rGel/BLyS were added in quadruplicate wells. After 96 hr, 75 μl of XTT labeling mixture was added to each well, after which the cells incubated for another 4 hr. The spectrophotometric absorbance was measured at 450 nm using an ELISA reader.

FIG. 11 shows the maximal tolerated dose (MTD) of rGel/BLyS. To obtain a MTD of rGel/BLyS, various concentrations of rGel/BLyS were injected into Balb/C mice for 5 consecutive days by i.v. tail vein and measured the body weight and the number of surviving mice.

FIG. 12 shows specificity of rGel/BLyS to BLyS receptor-expressing cells. (FIG. 14A) The receptor-binding activity moiety of BLyS component of the rGel/BLyS was determined using intact JeKo-1 and HL-60 cells by ELISA. (FIG. 14B) To examine the specific activity of rGel/BLyS against three BLyS receptor(s) expressing mantle cell lymphoma (MCL) cell lines, JeKo-1 MCL cell line was seeded (5×10³ cells/well) in flat-bottom 96-well microtiter plates and BLyS, rGel, CTP/rGel, or rGel/BLyS were added in quadruplicate wells. After 96 hr, 50 μl of XTT labeling mixture was added to each well, after which the cells incubated for another 4 hr or overnight. The spectrophotometric absorbance was measured at 450 nm using an ELISA reader.

FIGS. 13A-13B demonstrate competitive inhibition of rGel/BLyS on JeKo-1 cells. For competitive inhibition assay, JeKo-1 cells were seeded (5×10³ cells/well) in flat-bottom 96-well microtiter plates (Becton Dickinson) and pre-treated with 1 nM of BLyS, 50 nM of BLyS (FIG. 15A), 10 μg/ml of BAFF-R:Fc, 10 μg/ml of TACI:Fc, or 10 μg/ml of BCMA:Fc (FIG. 15B) for 2 hr, and then rGel, BLyS or rGel/BLyS was added in quadruplicate wells. After 96 hr, 50 μl of XTT labeling mixture (Roche) was added to each well, after which the cells incubated for another 4 hr or overnight. The spectrophotometric absorbance was measured at 450 nm using an ELISA reader.

FIGS. 14A-14C show the effects of rGel/BLyS on apoptotic pathways. (FIG. 14A) Microscopic appearance of JeKo-1 cells after treatment. JeKo-1 cells were treated with 100 pM BLyS, 100 pM rGel, or 100 pM rGel/BLyS. After 96 hr, JeKo-1 cells were assayed for apoptosis by TUNEL staining. (FIG. 14B) Apoptotic cells were counted in randomly selected fields (×200) and expressed as a percentage. To examine the effect of rGel/BLyS on apoptotic pathways, JeKo-1 or Granta 519 cells were seeded at 5×10⁵ cells/24-well plate and then treated with 100 pM BLyS, 100 pM rGel, or 100 pM rGel/BlyS. After treatment, cells were collected, washed, and lysed in 0.2 ml of lysis buffer. Cell lysates (50 μg) were fractionated by 8-15% SDS-PAGE and electrophoretically transferred to Immobilon-P nitrocellulose membranes. Membranes were blocked, and then probed with various antibodies (FIG. 14C). Secondary antibodies conjugated with horseradish peroxidase were used to visualize immunoreactive proteins using ECL detection reagent. Actin was used as a control for protein loading.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

I. Definitions

The term “conjugated” as used herein refers in some embodiments to the attachment of a BLyS molecule to a cytotoxic agent molecule. The attachment may be through any suitable methods in the art, although in specific aspects the attachment is via recombination, by a linker, and so forth. In particular aspects, an ionic association is employed, such as through the use of an avidin-biotin linkage, for example.

II. The Present Invention

The present invention provides compositions that are targeted to abnormally proliferating B-cells that display receptors for the BLyS polypeptide. Normally proliferating B cells, as well as other cell types, do not express BLyS receptors. The polypeptides of the present invention comprise a BLyS polypeptide that serves as a targeting domain, and a cytotoxic peptide that reduces or eliminates proliferation of the targeted cell(s). Such polypeptides have specific cytotoxic activity against abnormally proliferating B cells, and are thus useful for therapy against any B-cell proliferative disorder. Methods of designing and using such polypeptides in therapy are described herein.

III. BLys Proteinaceous Compounds

The present invention concerns targeted conjugated polypeptides, particularly those that confer a therapeutic benefit to a subject. In certain embodiments, the present invention concerns novel compositions comprising a proteinaceous molecule. In certain embodiments, it is contemplated that the proteinaceous compound may be modified through deletion, substitution, or addition of amino acid residues. In particular embodiments, the BLyS comprises a mutation that still allows the molecule to comprise activity to bind a BLyS receptor. The mutation may be present in the polynucleotide encoding the BLyS molecule. Exemplary BLyS mutants include a mutation at Cys146 (Chen et al., 2002; 2004; 2005), and in specific embodiments the mutation is to alanine or valine. BLyS mutants employed in the invention will retain the ability to target B cells, such as by retaining the ability to bind at least one BLyS receptor. A BLyS mutant may enhance any activity over a wild type BLyS, including the ability to bind a B cell, such as through a BLyS receptor.

Furthermore, a proteinaceous compound may include an amino acid molecule comprising more than one polypeptide entity. As used herein, a “proteinaceous molecule,” “proteinaceous composition,” “proteinaceous compound,” “proteinaceous chain” or “proteinaceous material” generally refers, but is not limited to, a peptide of from about 3 to about 100 amino acids, a polypeptide of greater than about 100 amino acids, and a polypeptide of greater than about 200 amino acids, or the full length endogenous sequence translated from a gene. All the “proteinaceous” terms described above may be used interchangeably herein. Furthermore, these terms may be applied to conjugated polypeptides or protein conjugates as well.

In certain embodiments the size of the at least one proteinaceous molecule may comprise, but is not limited to, about or at least 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 350, 400, 450, 500, 550, 600, 650, 700, 750, 800, 850, 900, 950, 1000, 1100, 1200, 1300, 1400, 1500, 1750, 2000, 2250, 2500 or greater amino molecule residues, and any range derivable therein.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Targeted conjugated polypeptides of the present invention may possess deletions and/or substitutions of amino acids; thus, proteins with a deletion, proteins with a substitution, and proteins with a deletion and a substitution are targeted conjugated polypeptides. In some embodiments these targeted conjugated polypeptides may further include insertions or added amino acids, such as linkers, for example. A “targeted fusion deleted protein” lacks one or more residues of the native protein, but possesses the specificity and/or activity of the native protein.

Substitutional or replacement variants typically contain the exchange of one amino acid for another at one or more sites within the protein and may be designed to modulate one or more properties of the polypeptide, particularly to increase its efficacy or specificity. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In addition to a deletion or substitution, a targeted fusion protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a native polypeptide are included, provided the biological activity of the protein is maintained. A targeted fusion protein may be biologically functionally equivalent to its native counterpart. For example, it may be functionally equivalent in terms of receptor binding ability. In other embodiments, the conjugated polypeptides of the present invention may have greater affinity for their receptors than their native counterparts.

The term “functionally equivalent codon” is used herein to refer to codons that encode the same amino acid, such as the six codons for arginine or serine, and also refers to codons that encode biologically equivalent amino acids (see Table 2, below).

TABLE 2

CODON TABLE

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, binding sites to substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 2 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

IV. Conjugated Polypeptides

The present invention further provides conjugated polypeptides, such as translated proteins, polypeptides and peptides, generally of the monoclonal type, that are linked to at least one agent to form a conjugate. The conjugation of the polypeptides of the present invention includes both chemical conjugation and “genetic conjugation,” such as recombinant fusion proteins. It is also contemplated that polypeptides of the present invention may be synthesized de novo using techniques known to one with skill in the art.

A. Peptide Synthesis

Conjugated polypeptides of the present invention may be synthesized. Peptide synthesis techniques are well known to those of skill in the art (Bodanszky et al., 1976) Peptide Synthesis, 1985; Solid Phase Peptide Synthelia, 1984); The Proteins, 1976. Appropriate protective groups for use in such syntheses will be found in the above texts, as well as in Protective Groups in Organic Chemistry, 1973. These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The peptides of the invention are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

B. Linkers/Coupling Agents

Multiple peptides or polypeptides may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin. Alternatively, peptides or polypeptides may be joined to an adjuvant.

Amino acids such as selectively-cleavable linkers, synthetic linkers, or other amino acid sequences may be used to separate proteinaceous moieties. Additionally, while numerous types of disulfide-bond containing linkers are known that can successfully be employed to conjugate the toxin moiety with the targeting agent, certain linkers will generally be preferred over other linkers, based on differing pharmacologic characteristics and capabilities. For example, linkers that contain a disulfide bond that is sterically “hindered” are to be preferred, due to their greater stability in vivo, thus preventing release of the toxin moiety prior to binding at the site of action. Furthermore, certain advantages in accordance with the invention will be realized through the use of any of a number of toxin moieties, including gelonin and a deglycosylated A chain of ricin.

It can be considered as a general guideline that any biochemical cross-linker that is appropriate for use in the present invention will also be of use in the present context, and additional linkers may also be considered.

Cross-linking reagents are used to form molecular bridges that tie together functional groups of two different molecules, e.g., a stablizing and coagulating agent. To link two different proteins in a step-wise manner, hetero-bifunctional cross-linkers can be used that eliminate unwanted homopolymer formation.

It is contemplated that cross-linkers may be implemented with the protein molecules of the invention. Bifunctional cross-linking reagents have been extensively used for a variety of purposes including preparation of affinity matrices, modification and stabilization of diverse structures, identification of binding sites, and structural studies. In the context of the invention, such cross-linker may be used to stabilize the polypeptide or to render it more useful as a therapeutic, for example, by improving the modified protein's targeting capability or overall efficacy. Cross-linkers may also be cleavable, such as disulfides, acid-sensitive linkers, and others. Homobifunctional reagents that carry two identical functional groups proved to be highly efficient in inducing cross-linking between identical and different macromolecules or subunits of a macromolecule, and linking of polypeptides to specific binding sites on binding partners. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. In instances where a particular polypeptide, such as gelonin, does not contain a residue amenable for a given cross-linking reagent in its native sequence, conservative genetic or synthetic amino acid changes in the primary sequence can be utilized.

Several methods are known in the art for the attachment or conjugation of a polypeptide to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the polypeptide (U.S. Pat. Nos. 4,472,509 and 4,938,948, each incorporated herein by reference). Polypeptides may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the polypeptide using linkers such as methyl-p-hydroxybenzimidate or N-succinimidyl-3-(4-hydroxyphenyl)propionate.

C. Imaging Agents

In some aspects of the invention, the BLyS polypeptide is conjugated to at least one imaging agent. Non-limiting examples of imaging agents which have been conjugated to polypeptides include enzymes, radiolabels, haptens, fluorescent labels, phosphorescent molecules, chemiluminescent molecules, chromophores, luminescent molecules, photoaffinity molecules, colored particles or ligands, such as biotin.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Intermediary functional groups which are often used to bind radioisotopes which exist as metallic ions to polypeptide are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as binding agents.

V. Therapeutic Agents

In particular aspects of the invention, the BLyS polypeptides of the present invention are conjugated to another molecule, which may be considered a therapeutic agent. In particular aspects, the therapeutic agent may be a cytotoxic agent, a radioisotope, a small molecule, a chemotherapeutic, a pro-apopotic agent, a natural product, an antibody, a cytokine, a chemokine, an angiogenic inhibitor, a regulator of programmed cell death, and so forth.

Some examples of therapeutic agents are discussed in the following text.

A. Cytotoxic Agents

The present invention utilizes a cytotoxic activity in a targeting molecule, and in specific embodiments the cytotoxic activity may be referred to as a toxin. Any toxin is suitable in the invention so long as it does not interfere with the targeting moiety of the conjugated polypeptide (for example) and so long as it at least slows down, if not completely inhibits, the proliferation of a targeted cell.

Ribosome-inhibitory toxins (RITs) are potent inhibitors of protein synthesis in eukaryotes. The enzymatic domain of these proteins acts as a cytotoxic n-glycosidase that is able to inactivate catalytically ribosomes once they gain entry to the intracellular compartment. This is accomplished by cleaving the n-glycosidic bond of the adenine at position 4324 in the 28srRNA, which irreversibly inactivates the ribosome apparently by disrupting the binding site for elongation factors. RITs, which have been isolated from bacteria, are prevalent in higher plants. In plants, there are two types: Type I toxins possess a single polypeptide chain that has ribosome inhibiting activity, and Type II toxins have an A chain, comparable to the Type I protein, that is linked by a disulfide bond to a B chain possessing cell-binding properties. Examples of Type I RITs are gelonin, dodecandrin, tricosanthin, tricokirin, bryodin, mirabilis antiviral protein, barley ribosome-inactivating protein (BRIP), pokeweed antiviral proteins (PAPs), saporins, luffins, and momordins. Type II toxins include ricin and abrin. Toxins may be conjugated or expressed as a fusion protein with any of the polypeptides discussed herein.

As part of the present invention, toxins such as ricin A-chain (Burbage, 1997), diphtheria toxin A (Massuda et al., 1997; Lidor, 1997), pertussis toxin A subunit, E. coli enterotoxin toxin A subunit, cholera toxin A subunit and Pseudomonas toxin c-terminal are suitable. It has demonstrated that transfection of a plasmid containing the fusion protein regulatable diphtheria toxin A chain gene was cytotoxic for cancer cells. Other toxins envisioned as useful for the present invention include Abrin, A/B heat labile toxins, Botulinum toxin, Helix pomatia, Jacalin or Jackfruit, Peanut agglutinin, Sambucus nigra, Tetanus, Ulex, and Viscumin.

It is envisioned that any of the above therapeutic agents may be conjugated to the polypeptides of the present invention. In some instances, it may be preferable to recombinantly express chimeric proteins including toxin portions of other proteins. In other instances, it may be preferable to chemically conjugate small molecule compounds to the converted internalizing polypeptides described herein.

In particular embodiments, the toxin comprises a mutation that still allows the molecule to comprise cytotoxic activity. The mutation may be present in the polynucleotide encoding the toxin.

B. Radiopharmaceuticals

A number of different radioactive substances, including radioisotopes, can be used in cancer therapy. Examples of radioactive isotopes for therapeutic applications include astatine²¹¹, ⁵¹chromium, ³⁶chlorine, ⁵⁷cobalt, ⁵⁸cobalt, copper⁶⁷, ¹⁵²europium, gallium⁶⁷, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m}, yttrium⁹⁰, lutetium¹⁷⁷, samarium¹⁵³, holmium⁶⁶, and actinium²²⁵, for example.

C. Chemopharmaceuticals

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy.

Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the Goodman & Gilman's “The Pharmacological Basis of Therapeutics” and in “Remington's Pharmaceutical Sciences”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

1. Alkylating Agents

Alkylating agents include but are not limited to: busulfan, chlorambucil, cisplatin, cyclophosphamide (cytoxan), dacarbazine, ifosfamide, mechlorethamine (mustargen), and melphalan. In specific aspects, troglitazaone can be used to treat cancer in combination with any one or more of these alkylating agents, some of which are discussed below.

2. Antimetabolites

Antimetabolites include but are not limited to, 5-fluorouracil (5-FU), cytarabine (Ara-C), fludarabine, gemcitabine, and methotrexate. Purine analogs and related compounds include, but are not limited to, mercaptopurine (6-mercaptopurine; 6-MP), thioguanine (6-thioguanine; TG) and pentostatin (2-deoxycoformycin). Mercaptopurine has been used in acute lymphocytic, acute granulocytic and chronic granulocytic leukemias. Thrioguanine has been used in the treatment of such cancers as acute granulocytic leukemia, acute lymphocytic leukemia and chronic lymphocytic leukemia. Pentostatin has been used in such cancers as hairy cell leukemias, mycosis fungoides and chronic lymphocytic leukemia. Mitotic inhibitors include, for example, docetaxel, etoposide (VP16), teniposide, paclitaxel, taxol, vinblastine, vincristine, and vinorelbine. Epipodophyllotoxins include such compounds as teniposide and VP16. Taxoids include but are not limited to compounds such as docetaxel and paclitaxel. Vinca alkaloids include such compounds as vinblastine (VLB) and vincristine.

3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include, but are not limited to, bleomycin, dactinomycin, daunorubicin, doxorubicin (Adriamycin), plicamycin (mithramycin) and idarubicin. Widely used in clinical setting for the treatment of neoplasms these compounds generally are administered through intravenous bolus injections or orally.

4. Hormones

Corticosteroid hormones are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer.

5. Miscellaneous Agents

Some chemotherapy agents do not qualify into the previous categories based on their activities. They include, but are not limited to, platinum coordination complexes, anthracenedione, substituted urea, methyl hydrazine derivative, adrenalcortical suppressant, amsacrine, L-asparaginase, and tretinoin.

D. Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

E. Peptide Mimetics

Another embodiment for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993), incorporated herein by reference. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting peptides disclosed herein, but with altered and even improved characteristics.

F. Antibodies

In certain embodiments, it may be desirable to make antibodies against the identified targeting peptides or their receptors. The appropriate targeting peptide or receptor, or portions thereof, may be coupled, bonded, bound, conjugated, or chemically-linked to one or more agents via linkers, polylinkers, or derivatized amino acids. This may be performed such that a bispecific or multivalent composition or vaccine is produced. It is further envisioned that the methods used in the preparation of these compositions are familiar to those of skill in the art and should be suitable for administration to human subjects, i.e., pharmaceutically acceptable. Preferred agents are the carriers are keyhole limpet hemocyanin (KLH) or bovine serum albumin (BSA).

The term “antibody” is used to refer to any antibody like molecule that has an antigen binding region, and includes antibody fragments such as Fab′, Fab, F(ab′)2, single domain antibodies (DABs), Fv, scFv (single chain Fv), and the like. Techniques for preparing and using various antibody based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

G. Cytokines and Chemokines

In certain embodiments, it may be desirable to couple specific bioactive agents to one or more targeting peptides for targeted delivery to an organ or tissue. Such agents include, but are not limited to, cytokines, chemikines, pro-apoptosis factors and anti-angiogenic factors. The term “cytokine” is a generic term for proteins released by one cell population which act on another cell as intercellular mediators. Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-.alpha. and -.beta.; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

Cytokines may be employed that have stimulatory activity or that have growth inhibitory activity, and in specific embodiments a composition of the invention utilizes one of each.

Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-alpha, MIP1-Beta, and IP-10. The skilled artisan will recognize that certain cytokines are also known to have chemoattractant effects and could also be classified under the term chemokines.

H. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl 2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl 2 protein now is recognized to be a member of a family of related proteins, which can be categorized as death agonists or death antagonists.

Subsequent to its discovery, it was shown that Bcl 2 acts to suppress cell death triggered by a variety of stimuli. Also, it now is apparent that there is a family of Bcl 2 cell death regulatory proteins which share in common structural and sequence homologies. These different family members have been shown to either possess similar functions to Bcl 2 (e.g., BclXL, BclW, BclS, Mcl-1, A1, Bfl-1) or counteract Bcl 2 function and promote cell death (e.g., Bax, Bak, Bik, Bim, Bid, Bad, Harakiri).

Exemplary pro-apoptotic agents also include TNF, any caspase, including caspase-3, caspase-7, caspase-6, caspase-9, or caspase 10a/b, for example, or any granzyme, including granzyme A or granzyme B, for example.

Non-limiting examples of pro-apoptosis agents contemplated within the scope of the present invention include gramicidin, magainin, mellitin, defensin, cecropin, or a combination or mixture thereof.

I. Angiogenic Inhibitors

In certain embodiments the present invention may concern administration of targeting peptides attached to anti-angiogenic agents, such as angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline.

J. Dosages

The skilled artisan is directed to “Remington's Pharmaceutical Sciences” 15th Edition, chapter 33, and in particular to pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA Office of Biologics standards.

Doses of any of the above therapeutic agents may be determined by one with skill in the art. In certain embodiments, appropriate doses may be about 0.1 mg/kg to about 0.3 mg/kg, or about 1.5 mg/m² to about 2 mg/m² can also be administered. Alternatively, about 0.1 mg/m², about 0.12 mg/m², about 0.14 mg/m², about 0.15 mg/m², about 0.2 mg/m², about 0.25 mg/m², about 0.5 mg/m², about 1.0 mg/m², about 1.2 mg/m², about 1.4 mg/m², about 1.5 mg/m², about 2.0 mg/m², about 2.5 mg/m², about 5.0 mg/m², about 6 mg/m², about 8 mg/m², about 9 mg/m², about 10 m mg/m² may be an appropriate dose.

VI. Fusion Proteins

Other embodiments of the present invention concern fusion proteins. These molecules generally have all or a substantial portion of a targeting peptide, linked at the N- or C-terminus, to all or a portion of a second polypeptide or proteion. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the instant invention comprise a targeting peptide linked to a therapeutic protein or peptide. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually and protein or peptide could be incorporated into a fusion protein comprising a targeting peptide. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

VII. Synthetic Peptides

Because of their relatively small size, the targeting peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

VIII. Protein Purification

While some of the embodiments of the invention involve recombinant proteins, the invention in some embodiments utilizes methods and processes for purifying proteins, including recombinant proteins. Generally, these techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC. In addition, the conditions under which such techniques are executed may be affect characteristics, such as functional activity, of the purified molecules.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur. A “substantially purified” protein or peptide

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, about 96%, about 97%, about 98%, about 99%, about 99.2%, about 99.4%, about 99.6%, about 99.8%, about 99.9% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

The use of a peptide tag in combination with the methods and compositions of the invention is also contemplated. A tag takes advantage of an interaction between two polypeptides. A portion of one of the polypeptides that is involved in the interaction may used as a tag. For instance, the binding region of glutathione S transferase (GST) may be used as a tag such that glutathione beads can be used to enrich for a compound containing the GST tag. An epitope tag, which an amino acid region recognized by an antibody or T cell receptor, may be used. The tag may be encoded by a nucleic acid segment that is operatively linked to a nucleic acid segment encoding a modified protein such that a fusion protein is encoded by the nucleic acid molecule. Other suitable fusion proteins are those with β-galactosidase, ubiquitin, hexahistidine (6×His), or the like.

IX. Nucleic Acid Molecules

In one aspect of the invention, a nucleic acid molecule encoding a conjugated polypeptide of the invention is utilized.

A. Polynucleotides Encoding Conjugated Polypeptides

The present invention concerns polynucleotides, isolatable from cells, that are free from total genomic DNA and that are capable of expressing all or part of a targeted fusion protein or polypeptide of the present invention. The polynucleotide may encode a native protein that may be manipulated to encode a targeted fusion protein. For example, a polynucleotide may encode multiple moieties such as a gelonin polypeptide that is covalently attached to a BLyS targeting polypeptide. As used in this application, the term “polynucleotide” refers to a nucleic acid molecule that has been isolated free of total genomic nucleic acid. Therefore, a “polynucleotide encoding a polypeptide” refers to a DNA segment that polypeptide-coding sequences isolated away from, or purified free from, total mammalian or human genomic DNA. Therefore, for example, when the present application refers to the function or activity of gelonin, “native gelonin polypeptide,” or “fusion gelonin polypeptide” that is encoded by a polynucleotide, it is meant that the polynucleotide encodes a molecule that has enzymatic activity of gelonin.

As used herein, the term “DNA segment” refers to a DNA molecule that has been isolated free of total genomic DNA of a particular species. Therefore, a DNA segment encoding a polypeptide refers to a DNA segment that contains wild-type, polymorphic, or mutant polypeptide-coding sequences yet is isolated away from, or purified free from, total mammalian or human genomic DNA. Included within the term “DNA segment” are a polypeptide or polypeptides, DNA segments smaller than a polypeptide, and recombinant vectors, including, for example, plasmids, cosmids, phage, viruses, and the like.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a particular polypeptide from a given species may be represented by natural variants that have slightly different nucleic acid sequences but, nonetheless, encode the same protein (see Table 1 above).

Similarly, a polynucleotide comprising an isolated or purified wild-type, polymorphic, or mutant polypeptide gene refers to a DNA segment including wild-type, polymorphic, or mutant polypeptide coding sequences and, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, conjugated polypeptides, and mutants. A nucleic acid encoding all or part of a native or modified polypeptide may contain a contiguous nucleic acid sequence encoding all or a portion of such a polypeptide of the following lengths: about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. It is contemplated that a single polynucleotide molecule may encode, 1, 2, 3, 4, 5, 6, 7, 8, 9, 10, or more different polypeptides (all or part).

In particular embodiments, the invention concerns isolated DNA segments and recombinant vectors incorporating DNA sequences that encode a targeted fusion polypeptide or peptide that includes within its amino acid sequence a contiguous amino acid sequence in accordance with, or essentially corresponding to a native polypeptide. Thus, an isolated DNA segment or vector containing a DNA segment may encode, for example, a fusion gelonin polypeptide that has the ribosome-inactivating activity and specificity of a native gelonin polypeptide, and that is operatively linked to a BLyS polynucleotide, for example of SEQ ID NO:4 or SEQ ID NO:5. The term “recombinant” may be used in conjunction with a polypeptide or the name of a specific polypeptide, and this generally refers to a polypeptide produced from a nucleic acid molecule that has been manipulated in vitro or that is the replicated product of such a molecule.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length polypeptide from any source or encode a truncated version of the polypeptide, for example a truncated gelonin polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In a non-limiting example, one or more nucleic acid constructs may be prepared that include a contiguous stretch of nucleotides identical to or complementary to the a particular gene, such as the toxin gelonin. A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

As used herein, the term “DNA segment” refers to a DNA molecule that has been isolated free of total genomic DNA of a particular species. Therefore, a DNA segment encoding a polypeptide refers to a DNA segment that contains wild-type, polymorphic, or mutant polypeptide-coding sequences yet is isolated away from, or purified free from, total mammalian or human genomic DNA. Included within the term “DNA segment” are a polypeptide or polypeptides, DNA segments smaller than a polypeptide, and recombinant vectors, including, for example, plasmids, cosmids, phage, viruses, and the like. The DNA segments used in the present invention encompass biologically functional equivalent polypeptides and peptides, for example, a modified functionally equivalent gelonin toxin or a modified functionally equivalent BLyS. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the cytotoxicity of the protein or to increase the efficacy of any treatment involving the protein.

B. Vectors

Native and modified polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference. In addition to encoding a modified polypeptide such as modified gelonin, a vector may encode non-modified polypeptide sequences such as a tag or targeting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targeting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996).

Promoters that are contemplated for use in expressing conjugated polypeptides, such as fusion proteins, of the present invention are shown below in Table 3.

TABLE 3
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et |
| | al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn et |
| | al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek et |
| | al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake et |
| | al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Choi et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et |
| | al., 1988; Feng et al., 1988; Takebe et al., 1988; Rosen et |
| | al., 1988; Berkhout et al., 1989; Laspia et al., 1989; Sharp |
| | et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

8. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

9. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

10. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

11. Viral Vectors

There are a number of ways in which expression vectors may be introduced into cells. In certain embodiments of the invention, the expression vector comprises a virus or engineered vector derived from a viral genome. The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986). Retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

X. Treatment Methods

The molecules of the invention, e.g., the protein conjugates, as envisioned for therapeutic use, are molecules that retain the specificity of the BLyS portion with the cytotoxic potential of the toxin or therapeutic agent.

It is contemplated that the polypeptides of the present invention are administered in therapeutically effective amounts to a patient in need thereof. The term “therapeutically effective” as used herein refers to the amount of a compound required to improve some symptom associated with a disease. For example, in the treatment of cancer, a compound that improves the cancer to any degree or arrests any symptom of the cancer would be therapeutically effective. For example, the improvement of the cancer may be inhibition of angiogenesis of a cancer cell and/or tissue, inhibition or retardation of cell growth, facilitation of cell death, or a combination thereof. A therapeutically effective amount of a compound is not required to cure a disease but will provide a treatment for a disease.

It is envisioned that polypeptides of the present invention are useful for treating B-cell chronic Lymphocytic leukemia/small lymphocytic lymphoma B-cell prolymphocytic leukemia, Immunocytoma/lymphoplasmacytic lymphoma (+/−Waldenstrom's macroglobulinemia), Mantle cell lymphoma, Marginal Zone B-cell Lymphoma of mucosa-associated lymphoid tissue (MALT) type, Splenic marginal zone B-cell Lymphoma (+/−villous Lymphocytes), Hairy cell leukemia, Diffuse large B-cell Lymphoma, Mediastinal (Thymic) large B-cell Lymphoma, Intravascular large B-cell Lymphoma, Burkitt Lymphoma, Plasma cell myeloma (multiple myeloma), Monoclonal gammopathy of undetermined significance (MGUS), Indolent myeloma, Smoldering myeloma, Osteosclerotic myeloma (POEMS syndrome), Plasma cell leukemia, Non-secretory myeloma, Plasmacytomas, Solitary plasmacytoma of bone, Extramedullary plasmacytoma, Waldenstrom's macroglobulinemia, Heavy Chain Disease (HCD), Immunoglobulin deposition diseases, Systemic light chain disease, and Primary amyloidosis

In preferred embodiments, the polypeptides of the present invention are used to treat humans.

XI. Combination Treatments/Cancer Therapies

In order to increase the effectiveness of a conjugated polypeptide of the present invention, or expression construct coding therefor, it may be desirable to combine these compositions with other agents effective in the treatment of B-cell proliferative disorders, such as anti-cancer agents. Indeed, in particular embodiments, the conjugated polypeptides of the present invention are employed with one or more chemotherapeutic agents, such as to render effective the chemotherapeutic agent on a cell, including a sensitive or a resistant cell. The conjugated polypeptides alone or in conjunction with one or more chemotherpeutic agents may be administered to an individual with a B-cell proliferative disorder in addition to another cancer therapy, such as radiation, surgery, gene therapy, and so forth.

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with another therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that conjugated polypeptides could be used similarly in conjunction with chemotherapeutic, radiotherapeutic, gene therapy, or immunotherapeutic intervention, in addition to other pro-apoptotic or cell cycle regulating agents.

The therapy may precede or follow the other agent treatment by intervals ranging from minutes to weeks. In embodiments where the other agent and conjugated polypeptide are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, wherein conjugated polypeptide therapy is “A” and the secondary agent, such as radio- or chemotherapy, for example, is “B”:

| | | | |
|---|---|---|---|
| A/B/A | B/A/B | B/B/A | A/A/B |
| A/B/B | B/A/A | A/B/B/B | B/A/B/B |
| B/B/B/A | B/B/A/B | A/A/B/B | A/B/A/B |
| A/B/B/A | B/B/A/A | B/A/B/A | B/A/A/B |
| A/A/A/B | B/A/A/A | A/B/A/A | A/A/B/A |

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

A. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a gene therapy in which a therapeutic polynucleotide is administered before, after, or at the same time as a conjugated polypeptide of the present invention. Delivery of a conjugate polypeptide in conjuction with a second vector encoding one of the following gene products will have a combined anti-hyperproliferative effect on target tissues. Alternatively, a single vector encoding both genes may be used. A variety of proteins are encompassed within the invention, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl 2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl 2 protein now is recognized to be a member of a family of related proteins, which can be categorized as death agonists or death antagonists.

Subsequent to its discovery, it was shown that Bcl 2 acts to suppress cell death triggered by a variety of stimuli. Also, it now is apparent that there is a family of Bcl 2 cell death regulatory proteins which share in common structural and sequence homologies. These different family members have been shown to either possess similar functions to Bcl 2 (e.g., BclXL, BclW, BclS, Mcl-1, A1, Bfl-1) or counteract Bcl 2 function and promote cell death (e.g., Bax, Bak, Bik, Bim, Bid, Bad, Harakiri).

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. The chimeric molecule of the present invention may be employed as neoadjuvant surgical therapy, such as to reduce tumor size prior to resection, or it may be employed as postadjuvant surgical therapy, such as to sterilize a surgical bed following removal of part or all of a tumor.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

XII. Pharmaceutical Compositions and Routes of Administration

The present invention contemplates nucleic acid molecules encoding fusion proteins. In some embodiments, pharmaceutical compositions are administered to a subject. Different aspects of the present invention involve administering an effective amount of an aqueous composition. Such compositions will generally be dissolved or dispersed in a pharmaceutically acceptable carrier or aqueous medium. Additionally, such compounds can be administered in combination with another treatment depending upon the disease or condition being treated. Treatment of lymphoma could include administration of chemotherapy, radiotherapy, immunotherapy, or hormones.

Those of skill in the art are well aware of how to apply gene delivery to in vivo and ex vivo situations. For viral vectors, one generally will prepare a viral vector stock. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² infectious viral particles to the patient. Similar figures may be extrapolated for liposomal or other non-viral formulations by comparing relative uptake efficiencies. Formulation as a pharmaceutically acceptable composition is discussed below.

The phrases “pharmaceutically acceptable” or “pharmacologically acceptable” refer to molecular entities and compositions that do not produce an adverse, allergic, or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

The active compounds of the present invention can be formulated for parenteral administration, e.g., formulated for injection via the intravenous, intramuscular, intrathoracic, subcutaneous, or even intraperitoneal routes. The preparation of an aqueous composition that contains a compound or compounds that increase the expression of an MHC class I molecule will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of therapeutic compositions according to the present invention will be via any common route so long as the target tissue is available via that route. In cases where the present invention is used as a viral vector, a primary consideration will be the desired location for the heterologous sequences carried by the vector. Routes of administration include oral, nasal, buccal, rectal, vaginal or topical. Alternatively, administration may be by orthotopic, intradermal subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery to the lung is contemplated. Volume of the aerosol is between about 0.01 ml and 0.5 ml. Similarly, a preferred method for treatment of colon-associated disease would be via enema. Volume of the enema is between about 1 ml and 100 ml. Direct intratumoral injection is the preferred mode, with continuous intratumoral perfusion a more specific embodiment.

In certain embodiments, it may be desirable to provide a continuous supply of therapeutic compositions to the patient. For intravenous or intraarterial routes, this is accomplished by drip system. For topical applications, repeated application would be employed. For various approaches, delayed release formulations could be used that provided limited but constant amounts of the therapeutic agent over and extended period of time. For internal application, continuous perfusion, for example with a viral vector carrying a heterologous nucleic acid segment, of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the injections are administered. It is believed that higher doses may be achieved via perfusion, however.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, Remington's Pharmaceutical Sciences, 1990). Some variation in dosage will necessarily occur depending on the condition of the subject being treated. The person responsible for administration will, in any event, determine the appropriate dose for the individual subject.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability, and toxicity of the particular therapeutic substance.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

As used herein, the term in vitro administration refers to manipulations performed on cells removed from an animal, including, but not limited to, cells in culture. The term ex vivo administration refers to cells that have been manipulated in vitro, and are subsequently administered to a living animal. The term in vivo administration includes all manipulations performed on cells within an animal.

In certain aspects of the present invention, the compositions may be administered either in vitro, ex vivo, or in vivo. In certain in vitro embodiments, an expression construct encoding a modified protein may be transduced into a host cell. The transduced cells can then be used for in vitro analysis, or alternatively for in vivo administration.

U.S. Pat. Nos. 4,690,915 and 5,199,942, both incorporated herein by reference, disclose methods for ex vivo manipulation of blood mononuclear cells and bone marrow cells for use in therapeutic applications.

In vivo administration of the compositions of the present invention are also contemplated. Examples include, but are not limited to, transduction of bladder epithelium by administration of the transducing compositions of the present invention through intravesicle catheterization into the bladder (Bass, 1995), and transduction of liver cells by infusion of appropriate transducing compositions through the portal vein via a catheter (Bao, 1996). Additional examples include direct injection of tumors with the instant transducing compositions, and either intranasal or intratracheal (Dong, 1996) instillation of transducing compositions to effect transduction of lung cells.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally and/or using aerosol, injection, infusion, continuous infusion, localized perfusion bathing target cells directly or via a catheter and/or lavage.

XIII. Kits of the Invention

Any of the compositions described herein may be comprised in a kit. In a non-limiting example, a BLyS conjugate and optionally an additional agent may be comprised in a kit. The kits will thus comprise, in suitable container means, a BLyS conjugate and optionally an additional agent of the present invention.

The kits may comprise a suitably aliquoted BLyS conjugate and optionally additional agent compositions of the present invention, whether labeled or unlabeled. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the BLyS conjugate, additional agent, and any other reagent containers in close confinement for commercial sale. Such containers may include injection or blow molded plastic containers into which the desired vials are retained.

Therapeutic kits of the present invention are kits comprising a BLyS conjugate and will generally contain, in suitable container means, a pharmaceutically acceptable formulation thereof. The kit may have a single container means, and/or it may have distinct container means for each compound.

When the components of the kit are provided in one and/or more liquid solutions, the liquid solution is an aqueous solution, with a sterile aqueous solution being particularly preferred. The BLyS conjugate composition(s) may also be formulated into a syringeable composition. In this case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate composition to and/or within the body of an animal. Such an instrument may be a syringe, pipette, forceps, and/or any such medically approved delivery vehicle.

Thus, in specific embodiments of the invention, the composition is comprised in a pharmaceutically acceptable carrier and/or is suitably aliquoted for delivery to an individual.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Design of the rGelonin/BLys Polypeptide

The cDNA encoding human BLys and recombinant gelonin were fused together by using the splice overlap extension PCR (OE-PCR) method (Higuchi et al. 1988) with BLys and recombinant gelonin DNA as templates. Orientation A (BLys-rGel) or orientation B (rGel-BLys) fusion proteins were generated by the OE-PCR method using the entire coding region of the BLys and recombinant gelonin as DNA templates to amplify individual gene fragments. To construct BLys-rGel, an upstream OE-PCR fragment encoding the enterokinase digestion site and the restriction enzymes KPN I was amplified from the N-terminal portion of the BLys gene using the oligonuceotide primers PET BLyS For, (5′-GGCGGAAGCGGTACCGACGACGACGACAAGGCCGTTCAGGGTCCA-3′ SEQ ID NO: 12), and BLys Bac Link, (5′-GCTCCCGCCTCCCCCCAGCAGTTTCAATGC-3′ SEQ ID NO:13). An adjoining downstream OE-PCR fragment encoding a G₄S linker and a restriction enzyme Xho I was amplified from the recombinant gelonin gene using the oligonucleotide primers Link RGel, (5′-GGGGGAGGCGGGAGCGGCCTGGACACCGTG-3′ SEQ ID NO:14), and RGel Bac, (5′-GCTCGTGTCGACCTCGAGTCATTATTTAGGATCTTTATC-3′ SEQ ID NO:15). The upstream and downstream OE-PCR fragments were then reassembled as a full-length fusion BLys-rGel gene encoding the fusion protein by an additional PCR step using a pair of oligonucleotide primers PET BLys For and RGel Bac flanking the 5′- and 3′-end (see above). The final PCR fragment was purified and cleaved with KPN I and Xho I restriction endonucleases and then cloned into the Novagen expression vector pET-32a that utilizes the T7 promoter for the transcriptional control of the inserted fusion gene. The fusion BLys-rGel gene construct was verified by DNA sequencing before protein expression. For the orientation B, RGel-BLys, procedures described above were employed in the DNA manipulations using the oligonucleotide primers PET Gel For, (5′-AGCCCAGATCTGGGTACCGACGACGACGACAAGGGCCTGGACACCGTGAGC-3′ SEQ ID NO:16); RGel Bac Link, (5′-GCTCCCGCCTCCCCCTTTAGGATCTTT-3′ SEQ ID NO:17) for the upstream fragement, and BLys For, (5′-GGGGGAGGCGGGAGCGCCGTTCAGGGTCCA-3′ SEQ ID NO:18); BLys Rev, (5′-GCCGTCGACCTCGAGTCATTACAGCAGTTTCAATGC-3′ SEQ ID NO:19) for the downstream fragment. The final gene fusion fragment was amplified by an additional PCR step using the oligonucleotide primers PET Gel For and BLys Rev. The constructs were then transformed into Escherichia coli strain AD494(DE3)pLysS for expression of the fusion protein.

### Example 2

### Chemical Conjugation of BLys to rGelonin and Purification of BLys/rGel Chemical Conjugate

Recombinant gelonin (rGel) containing an extra cysteine residue for site-specific conjugation was generated as described previously and conjugated to the BLyS using SPDP as described previously (Rosenblum et al., 1991; Mujoo et al. 1995; Rosenblum et al., 1996). The chemical conjugate, BLyS/rGel, was purified using fast-protein liquid chromatography system (Pharmacia, New York, N.Y.) combining gel permeation (S-200) and affinity (Blue Sepharose) chromatography. Purity of the BLyS/rGel conjugate was assessed by SDS-PAGE and Western Blot Analysis.

### Example 3

### Cytotoxicity Studies

Table 4, below, outlines the results of various cytotoxicity studies performed with rGel/BLyS fusion proteins.

TABLE 4
Expression of BLyS, BAFF-R, TACI, BCMA, and comparative IC₅₀ values of the rGel/BLyS
against various types of cell lines

| | Cell type | BLyS | BAFF-R | TACI | BCMA | IC₅₀ (nM) | | |
|---|---|---|---|---|---|---|---|---|
| Cell line | (bp) | 313 | 256 | 196 | 285 | rGel | rGel/BLyS | Targeting index* |
| Jurkat | acute T cell leukemia | + | ++ | − | − | 3,000 | 1,500 | 2.0 |
| KBM-5 | myeloid leukemia | + | + | − | − | 250 | 70 | 3.6 |
| THP-1 | acute monocytic | + | ++ | − | − | 110 | 30 | 3.7 |
| | leukemia | | | | | | | |
| HL-60 | acute promyelocytic | + | ++ | − | − | 1,000 | 300 | 3.3 |
| | leukemia | | | | | | | |
| IM-9 | multiple myeloma | + | +++ | + | + | 700 | 200 | 3.5 |
| MM1.S | multiple myeloma | + | + | + | + | 600 | 220 | 2.7 |
| MM1.R | multiple myeloma | + | + | + | + | 1,000 | 280 | 3.6 |
| RPMI 8226 | plasmacytoma | + | ++ | + | + | 280 | 10 | 28 |
| | myeloma | | | | | | | |
| 8226/LR-5 | plasmacytoma | + | ++ | + | + | 200 | 110 | 1.8 |
| | myeloma | | | | | | | |
| JEKO | mantle cell lymphoma | + | +++ | + | + | 200 | 0.002 | 100,000 |
| SP53 | mantle cell lymphoma | + | +++ | + | + | 60 | 0.001 | 60,000 |
| Mino | mantle cell lymphoma | + | +++ | + | + | 35 | 0.005 | 7,000 |
| Granta | mantle cell lymphoma | + | ++ | + | + | 1,500 | 700 | 2.1 |
| BCL-1 | mouse B lymphoma | N.D. | N.D. | N.D. | N.D. | 150 | 0.0008 | 187,500 |

Targeting index represents IC₅₀ of rGel/IC₅₀ of rGel/BLyS.
N.D. represents not determined.

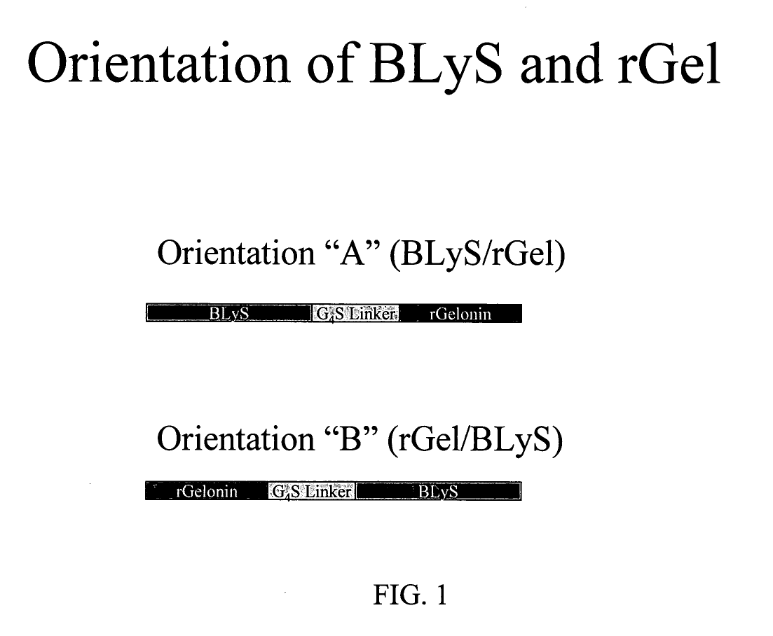

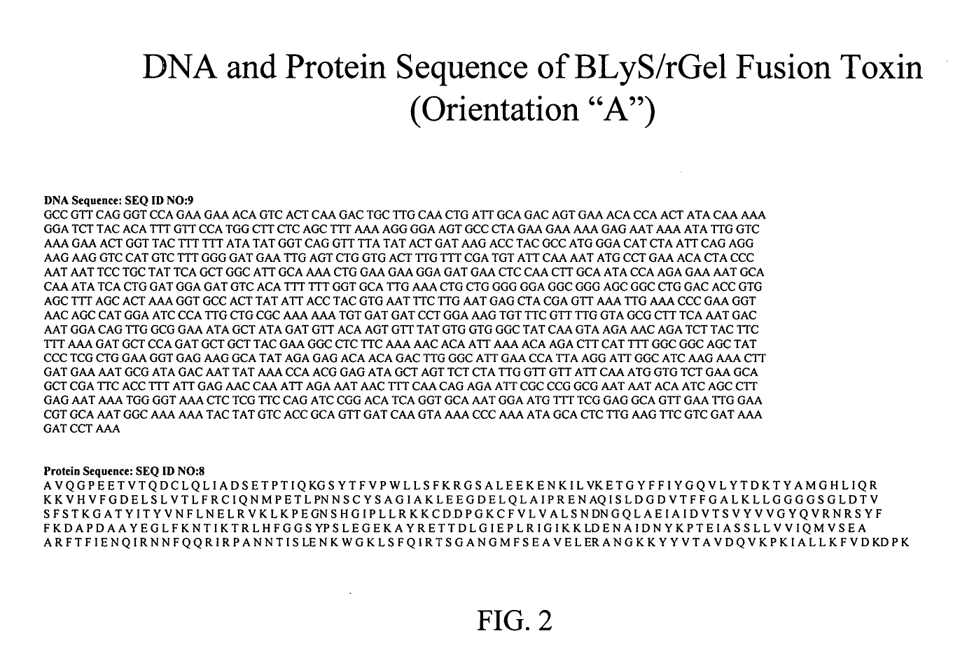

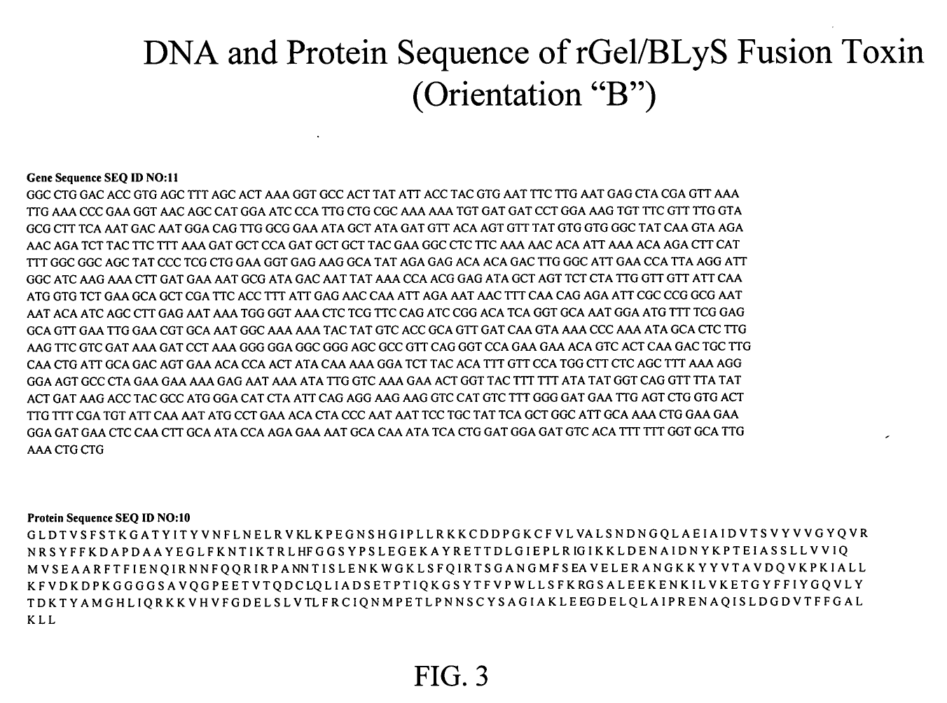

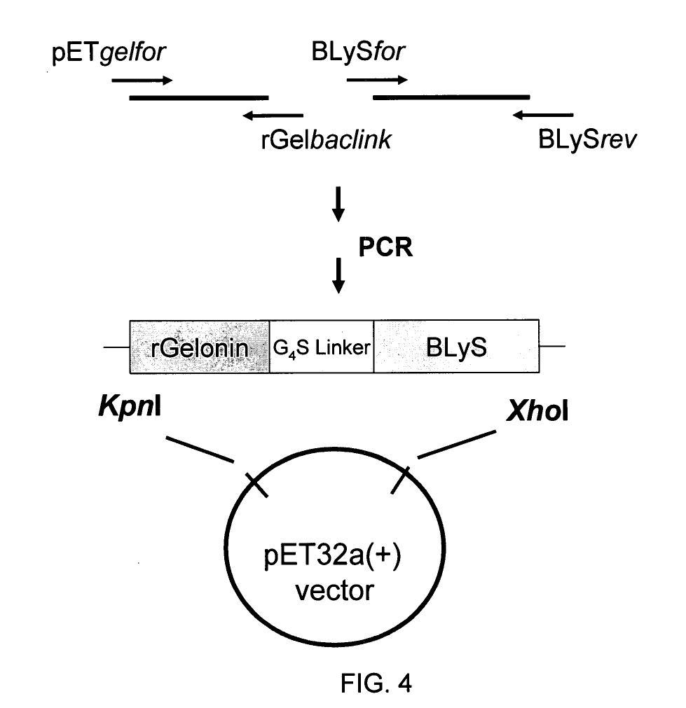

FIG. 1 provides an illustration of the orientation of BLyS and rGel. FIG. 2 shows exemplary DNA (SEQ ID NO:9) and protein sequences (SEQ ID NO:8) of an exemplary BLyS/rGel fusion toxin, and FIG. 3 shows exemplary DNA (SEQ ID NO:11) and protein sequences (SEQ ID NO:10) of an exemplary rGel/BLyS fusion toxin. FIG. 4 illustrates construction of an exemplary fusion toxin rGel/BLyS.

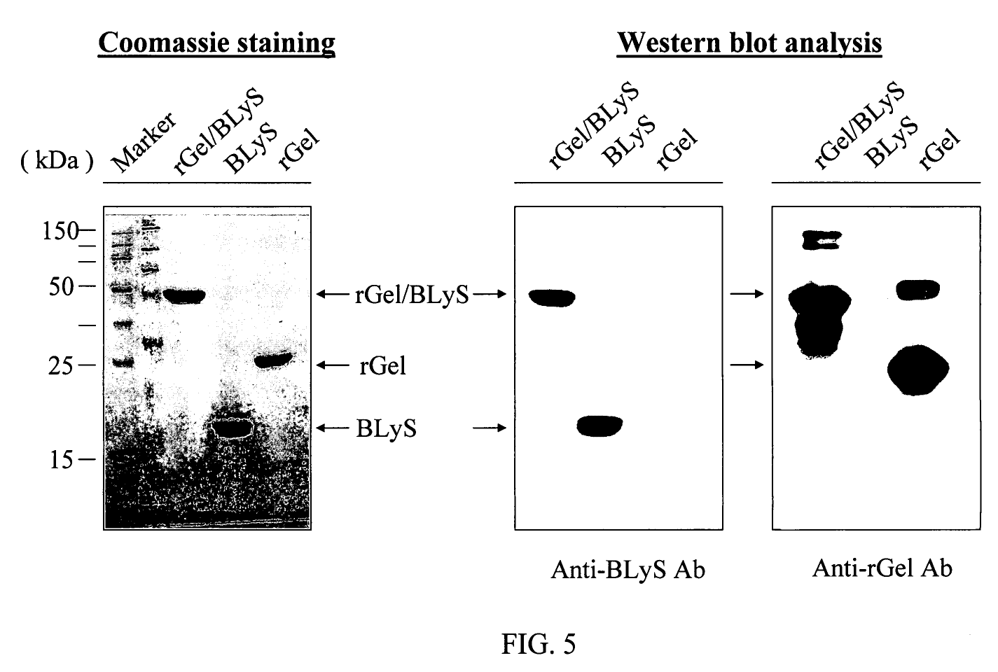

FIG. 5 shows the purification of rGel/BLyS fusion toxin. As shown in the Coomassie-stained SDS-PAGE analysis of the rGel/BLyS fusion toxin, the Mᵣ of rGel/BLyS was 45 kDa, demonstrating a 1:1 molar ratio of BLyS and rGel (left panel). Western blot analysis using anti-gelonin antibody or anti-BlyS antibody demonstrated that the rGel/BLyS fusion toxin contained toxin and BLyS component in the fusion toxin (right panel).

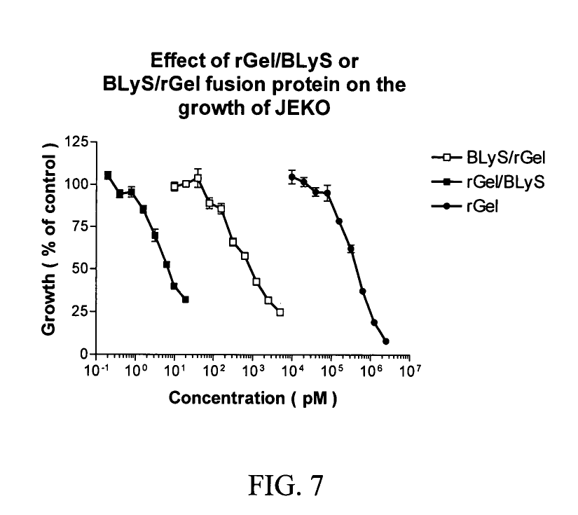

FIG. 7 is a comparison of the cytotoxic activity of rGel/BLyS and BLyS/rGel fusion toxin against JEKO mantle cell line. JEKO mantle cell lines were seeded (5×10³/well) in flat-bottom 96-well microtiter plates and rGel, rGel/BLyS, or BLyS/rGel were added in quadruplicate wells. After 96 hr, 75 μl of XTT labeling mixture was added to each well, after which the cells incubated for another 4 hr. The spectrophotometric absorbance was measured at 450 nm using an ELISA reader.

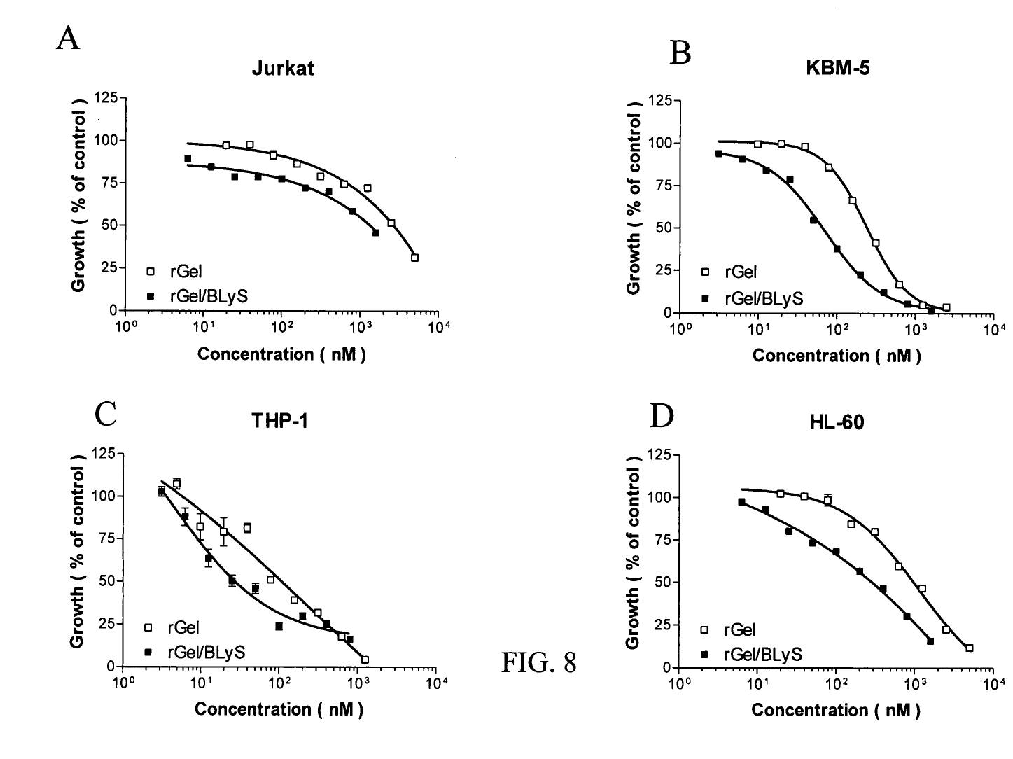

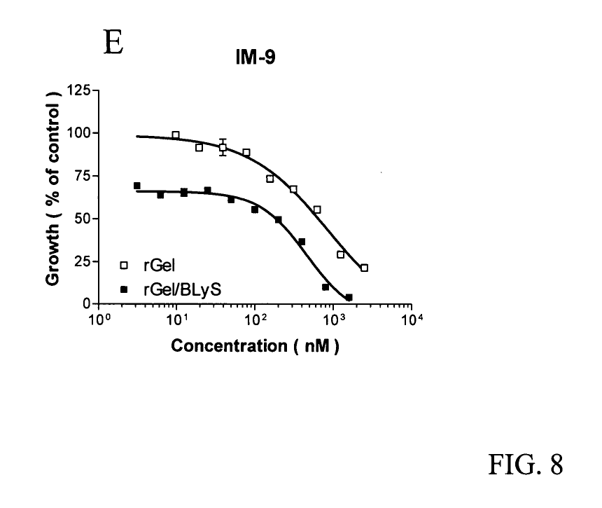

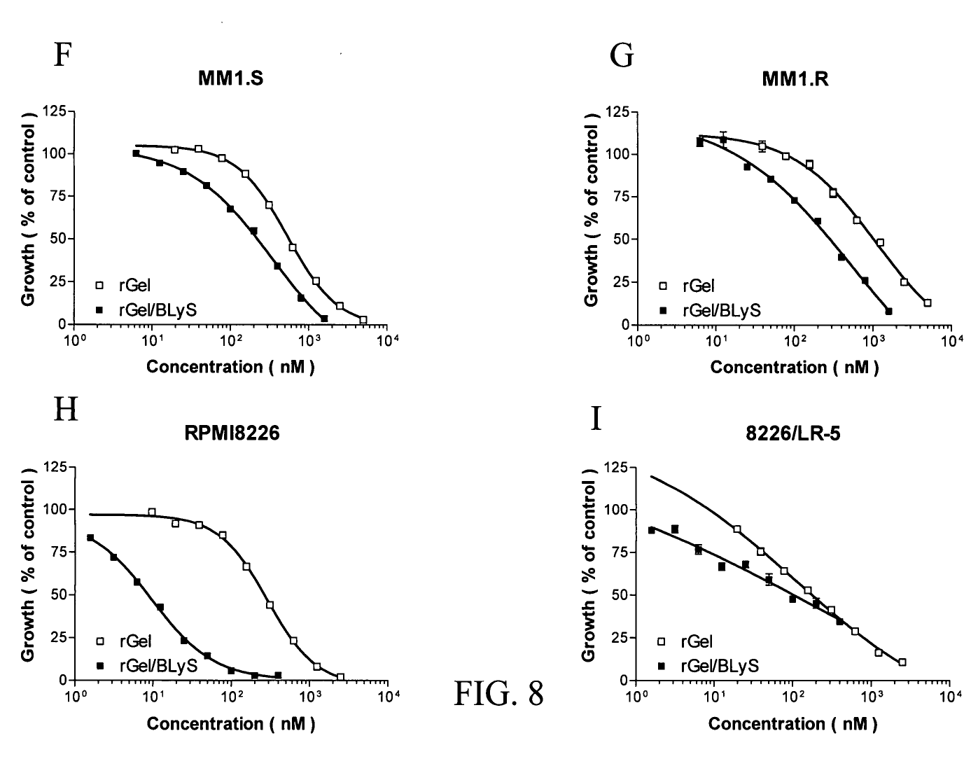

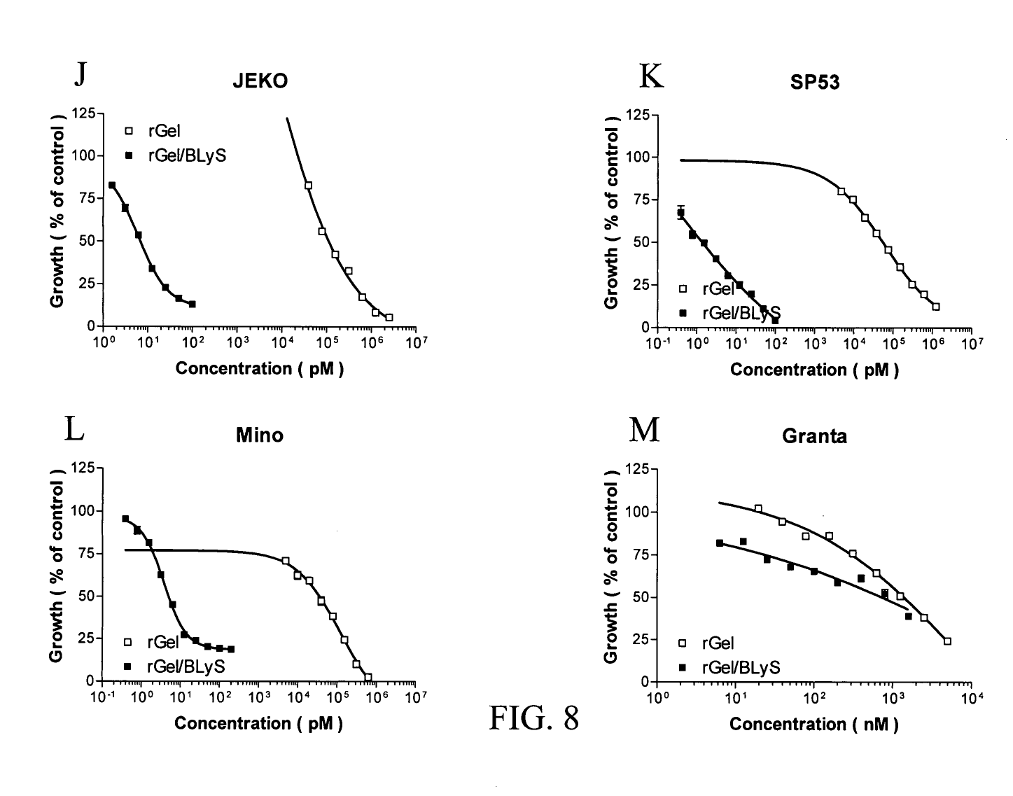

FIGS. 8A-8M show dose-response curves of rGel/BLyS fusion toxin against various tumor cell lines. Jurkat (8A), KBM-5 (8B), THP-1 (8C), HL-60 (8D), IM-9 (8E), MM1.S (8F), MM1.R (8G), RPMI8226 (8H), 8226/LR-5 (8I), JEKO (8J), SP53 (8K), Mino (8L), and Granta (8M). Thirteen tumor cell lines were seeded (5×10³/well) in flat-bottom 96-well microtiter plates and rGel, or rGel/BLyS were added in quadruplicate wells. After 96 hr, 75 μl of XTT labeling mixture was added to each well, after which the cells incubated for another 4 hr. The spectrophotometric absorbance was measured at 450 nm using an ELISA reader.

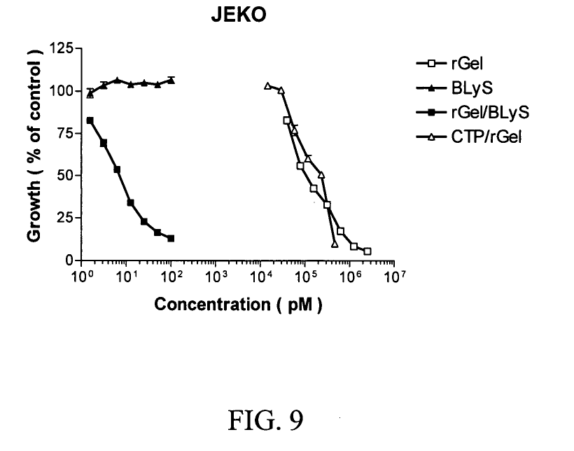

FIG. 9 shows the specificity of rGel/BLyS fusion toxin against BLyS receptor expressing JEKO mantle cell lines. JEKO mantle cell lines were seeded (5×10³/well) in flat-bottom 96-well microtiter plates and BLyS, rGel, CTP/rGel, and rGel/BLyS were added in quadruplicate wells. After 96 hr, 75 μl of XTT labeling mixture was added to each well, after which the cells incubated for another 4 hr. The spectrophotometric absorbance was measured at 450 nm using an ELISA reader.

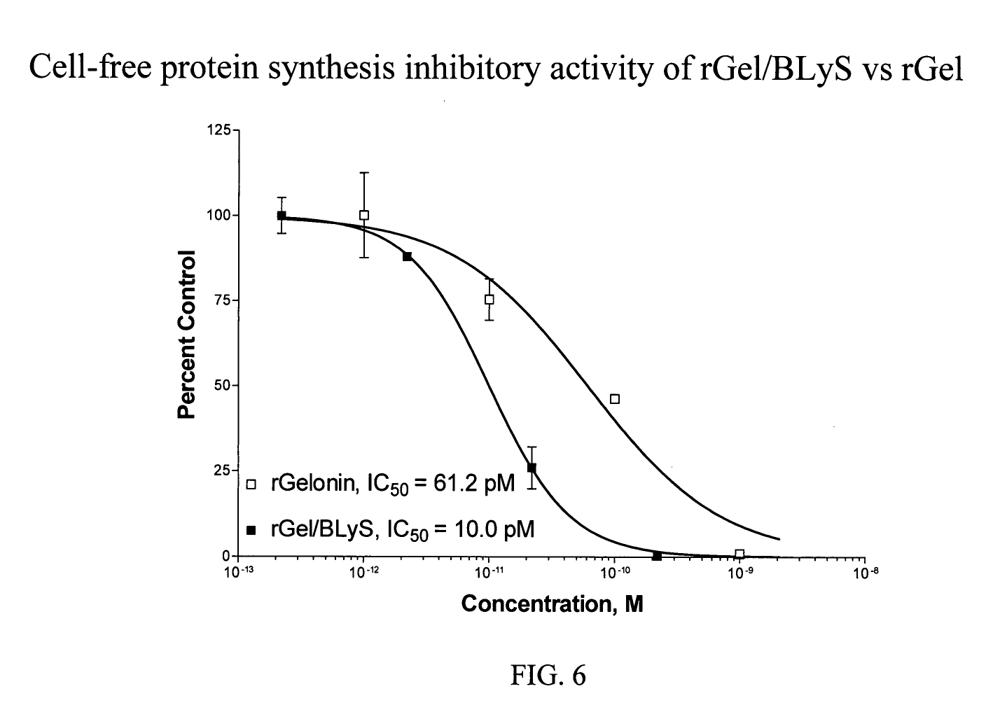

FIG. 6 shows cell-free protein synthesis inhibitory activity of the rGel/BLyS fusion toxin. To examine the n-glycosidic activity of the rGel component of the rGel/BLyS fusion toxin, this material was added to an in vitro protein translation assay using [³H]-leucine incorporation by isolated rabbit reticulocytes. Inhibition curves for the rGel/BLyS fusion toxin and native rGel were compared.

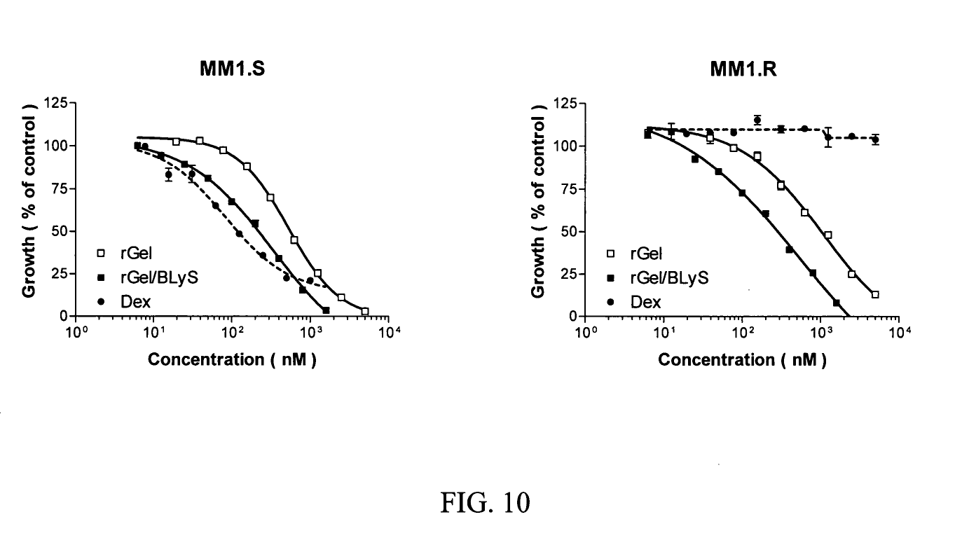

FIG. 10 is a dose-response curves of rGel/BLyS fusion toxin against Dexamethasone-sensitive (MM1.S) and -resistant (MM1.R) multiple myeloma cell lines. MM1.S and MM1.R cell lines were seeded (5×10³/well) in flat-bottom 96-well microtiter plates and rGel, Dex, or rGel/BLyS were added in quadruplicate wells. After 96 hr, 75 μl of XTT labeling mixture was added to each well, after which the cells incubated for another 4 hr. The spectrophotometric absorbance was measured at 450 nm using an ELISA reader.

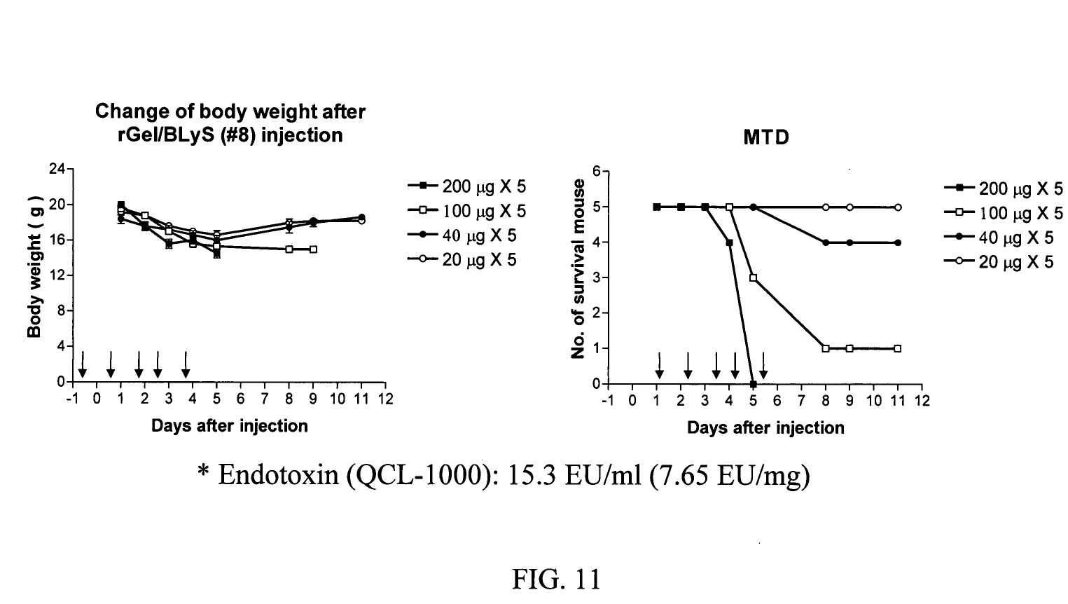

FIG. 11 shows the maximal tolerated dose (MTD) of rGel/BLyS. To obtain a MTD of rGel/BLyS, various concentrations of rGel/BLyS were injected into Balb/C mice for 5 consecutive days by i.v. tail vein and the body weight and the number of surviving mice were measured.

### Example 5

### Construction, Expression, and Purification of the Fusion Toxin rGel/BLyS

The present inventors constructed the rGel/BLyS fusion construct orienting rGel at the N-terminus followed by a G4S peptide tether to the BLyS molecule using a splice overlap extension PCR method (Ho et al., 1989) (FIG. 4). The fusion construct was then ligated into the Kpn I and Xho I sites of the pET-32a(+) vector, transformed into E. coli AD494 (DE3) strain. The rGel/BLyS was expressed and purified using immobilized metal ion affinity chromatography (Amersham). After enzymatic removal of the 20 kDa His-tag, the purified rGel/BLyS migrated on SDS-PAGE as a monomer at the expected molecular weight of 45.5 kDa under reducing conditions. The rGel/BLyS was also immunoreactive with antibodies to BLyS and rGel, thus demonstrating the presence of both toxin and BLyS components in the fusion construct (FIG. 5).

### Example 6

### Biological Activity of the rGel Component of the Fusion Toxin rGel/BLyS

The biological activity of toxins can be severely compromised when conjugated or fused to other proteins. To examine the n-glycosidic activity of the rGel component of the fusion toxin rGel/BlyS, this material was added to a cell-free protein synthesis assay using leuciferase production. Inhibition curves for the rGel/BLyS and native rGel were compared. The calculated IC50 values for rGel/BLyS and rGel were found to be 10 pM and 61 pM, respectively. Therefore, the results suggest that the enzymatic activity of the rGel component of rGel/BLyS was slightly more active that that of free rGel (FIG. 6). A similar finding was also reported for a fusion construct of VEGF₁₂₁ and rGel. The VEGF₁₂₁/rGel homodimer was shown to have an increased specific activity compared to rGel toxin itself (Veenendaal et al., 2002). These data indicate that multimerization of the rGel component may allow cooperativity between adjacent toxin molecules, in certain embodiments of the invention.

### Example 7

### BLyS, BAFF-R, TACI, and BCMA Expression and Response to rGel/BLyS

The present inventors examined the expression profile of the BLyS ligand and its three receptors by RT-PCR analysis using a panel of leukemia, myeloma, and mantle cell lymphoma cell lines. Thirteen cell lines (Jurkat, KBM-5, THP-1, HL-60, IM-9, MM1.S, MM1.R, RPMI 8226, 8226/LR-5, JeKo-1, SP53, Mino, and Granta 519) expressed BLyS and BAFF-R whereas TACI and BCMA were expressed in 9 exemplary cell lines tested with the exception of 4 leukemia cell lines (Jurkat, KBM-5, THP-1, and HL-60).

It was next examined whether a correlation existed between the expression levels of BLyS receptor (s) and sensitivity to rGel/BLyS. The comparative IC50 values of rGel/BLyS were examined against 13 exemplary cell lines including leukemia, myeloma, and mantle cell lymphoma. The ratio of IC₅₀ values of rGel to rGel/BLyS was calculated for each cell type. This ratio (targeting index) represents the ability of the BLyS component of the rGel/BLyS to mediate the delivery of the toxin component to the target cell cytoplasm. As summarized in Table 4, Jurkat, KBM-5, THP-1, HL-60, IM-9, MM1.S, MM1.R, 8226/LR-5, and Granta 519 showed a targeting index between 2 and 4 whereas 3 MCL cell lines (JeKo-1, SP53, and Mino) expressing BAFF-R, TACI and BCMA were highly sensitive to the rGel/BLyS and showed targeting index between 7,000 and 100,000. The MCL cell line JeKo-1 was found to be the most sensitive to rGel/BLyS (targeting index=100,000). The present inventors were unable to find a direct correlation between the expression levels of BLyS receptor(s) and sensitivity to rGel/BLyS.

### Example 8

### Binding Activity of rGel/BLyS

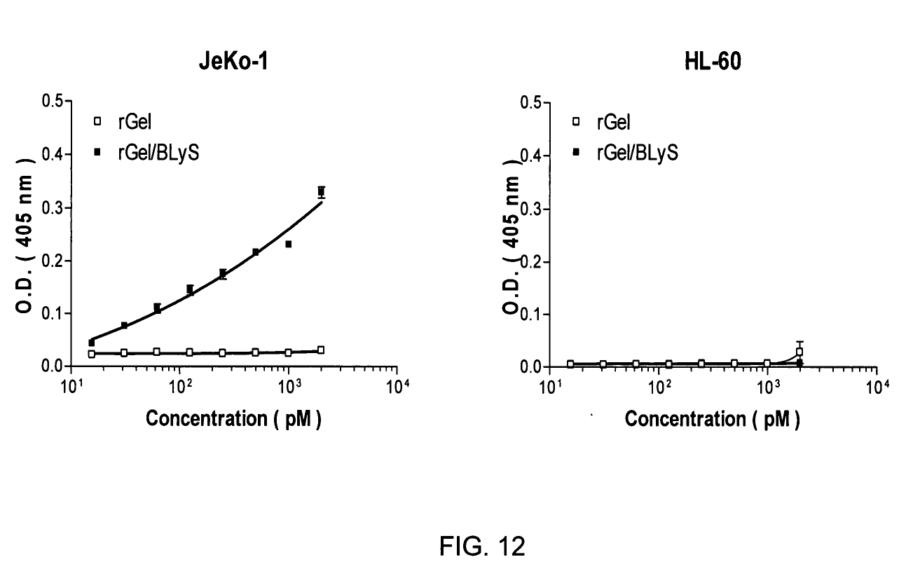

The cell-binding activity of the BLyS component of the rGel/BLyS was compared using intact JeKo-1 and HL-60 cell lines. The fusion toxin rGel/BLyS demonstrated specific binding activity to JeKo-1 cells expressing all three BLyS receptors whereas rGel did not bind to JeKo-1 and HL-60 cells. Interestingly, rGel/BLyS did not bind to HL-60 cells expressing only BAFF-R as assessed by PCR (FIG. 12).

### Example 9

### Specificity of rGel/BLyS

To assess the specificity of rGel/BLyS against BLyS receptor expressing cells, we treated JeKo-1 cells with BLyS itself, free rGel, CTP/rGel (non-B cell targeting chemical conjugate), or rGel/BLyS. BLyS itself proliferated the cell growth whereas non-B-cell targeting conjugate CTP/rGel showed a similar IC₅₀ of free rGel. However, rGel/BLyS was very cytotoxic to JeKo-1 cells expressing three BLyS receptors (FIG. 9).

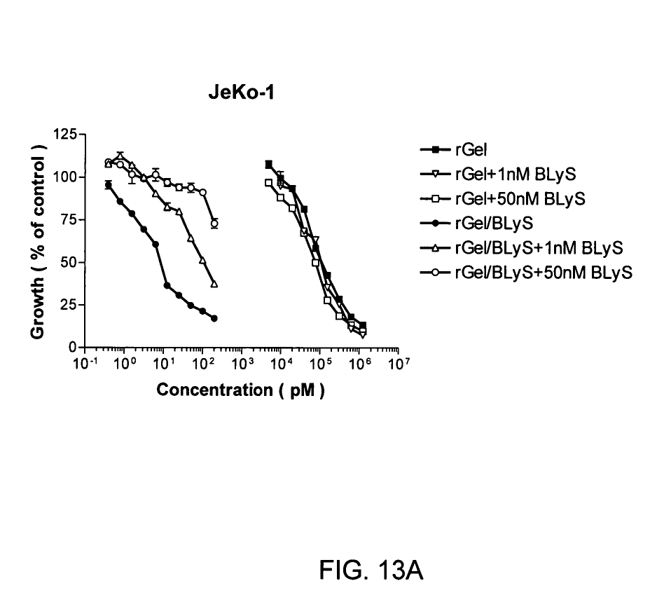

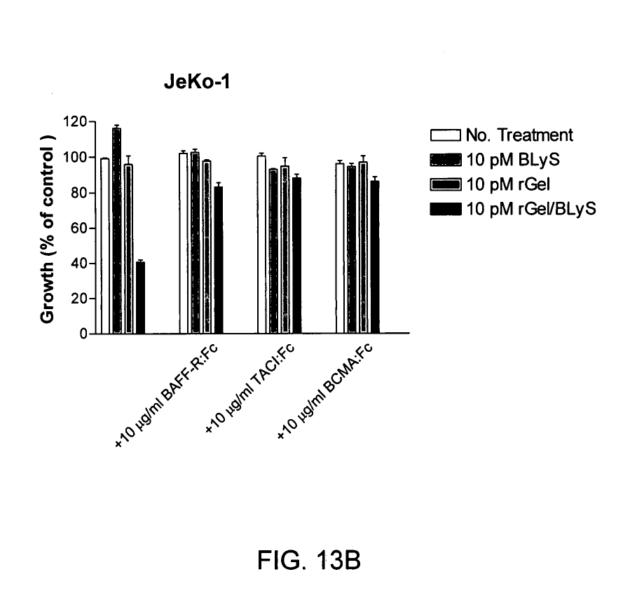

Pre-treatment of BLyS showed a shift in the dose-response curve in rGel/BLyS-treated JeKo-1 cells but not in rGel-treated JeKo-1 cells (FIG. 13A). In addition, pre-treatment of BAFF-R:Fc, TACI:Fc, or BCMA:Fc blocked the cytotoxic activity of rGel/BLyS in JeKo-1 cells but not in rGel- and BLyS-treated JeKo-1 cells (FIG. 13B). These data demonstrate that the cytotoxic effects of rGel/BLyS appear to be BLyS receptor-mediated. In addition, it appears that any one of the three receptors may be effective in mediating the cellular cytotoxic effects of the fusion toxin.

### Example 10

### Internalization of rGel/BLyS into JeKo-1 Mantle Cell Lymphoma (MCL) Cell Line

Internalized rGel/BLyS was detected using rabbit anti-rGel antibody. The rGel moiety of rGel/BLyS was observed in cytoplasm and nucleus after 1 hr exposure to the rGel/BLyS, thus demonstrating that the fusion toxin rGel/BLyS is capable of efficient cell binding through BLyS binding to the BLyS receptors for rapid internalization and delivery of the rGel toxin to the cytoplasm and nucleus of JeKo-1 cells.

### Example 11

### Effects of rGel/BLyS on Apoptotic Pathways

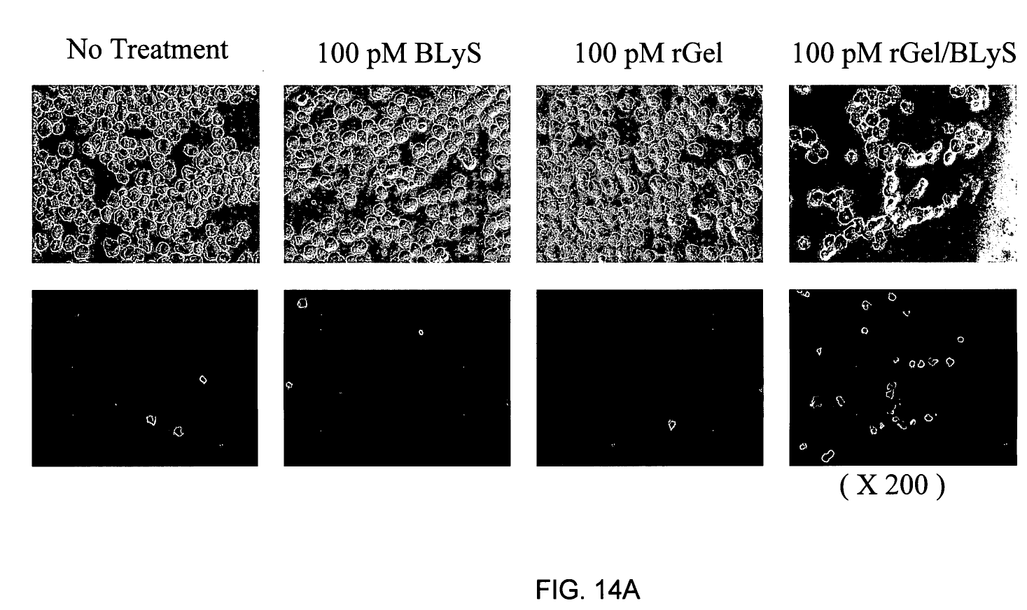

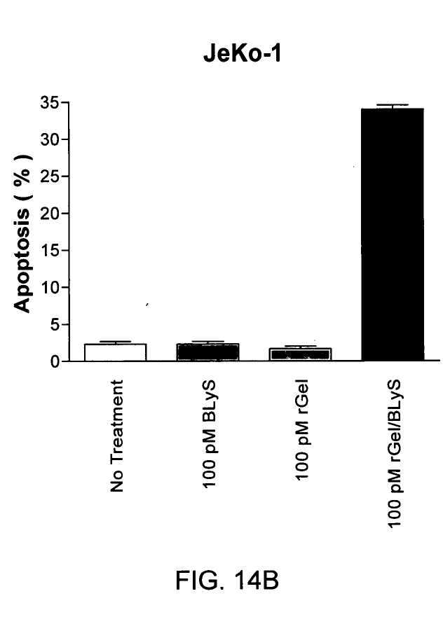

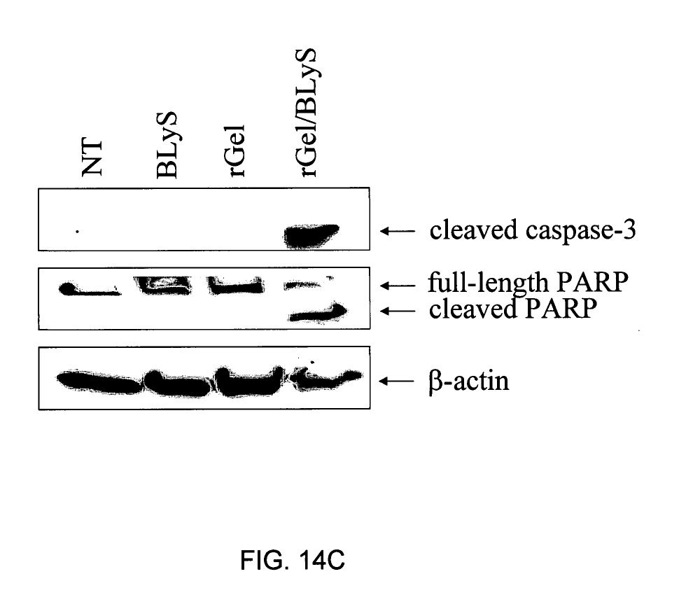

To determine whether the cytotoxic effect of rGel/BLyS was associated with an apoptotic mechanism, JeKo-1 cells were treated with 100 pM BLyS, 100 pM rGel, or 100 pM rGel/BLyS. After 96 hr, JeKo-1 cells were assayed for apoptosis by TUNEL staining. The rGel/BLyS-treated JeKo-1 cells showed 34% apoptotic cell death, whereas rGel treatment did not induce apoptosis (FIGS. 14A and 14B).

The caspase series of proteins are known to be a central mediator of the cellular apoptotic process. To determine whether caspase-3 was activated in JeKo-1 cells during rGel/BLyS-induced cell death, we examined the cleavage of caspase-3 and its substrate poly (ADP)-ribose polymerase (PARP). Treatment with BLyS or rGel had no effect on caspase-3 and PARP cleavage whereas treatment with rGel/BLyS resulted in cleavage of caspase-3 and PARP at 96 hr (FIG. 14C).

### Example 12

### Significance of the Present Invention

BLyS is an essential growth factor promoting peripheral B cell development and the growth stimulatory effects of BLyS are mediated by three cell-surface receptors designated BAFF-R, TACI and BCMA (Thompson et al., 2001; von Bulow and Bram, 1997; Laabi et al., 1992). These reports suggest that BLyS appears to be expressed in variable patterns in B-CLL specimens and may describe a subset of patients with inherent resistance to therapeutic agents. The inventors therefore chose BLyS as a targeting ligand for the specific delivery of rGel toxin to tumor cells expressing one or more of the receptors for BLyS. The inventors chose the rGel/BLyS orientation over the BLyS/rGel orientation for the fusion construct because unpublished data indicated that an unhindered C terminus for the BLyS molecule was important for trimerization and receptor recognition. Further studies with an inactive BLyS/rGel fusion construct have confirmed the observations in this regard.

To find a potential correlation between the cellular expression levels of BLyS receptor and sensitivity to rGel/BLyS, the present inventors examined the expression levels of BLyS and its three receptors and comparative IC₅₀ values of rGel/BLyS and rGel against various human cell lines including leukemia, multiple myeloma, mantle cell lymphoma, and mouse B lymphoma cell lines. BCL-1 mouse B lymphoma cell lines showed the highest targeting index (187,500). Kanakaraji et al. (2001) reported that BCL-1 cells have 4,800 binding site/cell whereas IM-9 cells have 3,200 binding site/cell. The responsiveness of BCL-1 cells to rGel/BLyS may also be related to the total number of BLyS receptors. Three MCL cell lines (JeKo-1, SP53, and Mino) expressing BAFF-R, TACI and BCMA were highly sensitive to the rGel/BLyS and showed targeting index between 7,000 and 100,000 whereas the Granta 519 MCL cell line showed a targeting index between 2 and 4 even though Granta 519 cells express all three receptors for BLyS at levels which are approximate to the expression on JeKo-1 cells as assessed by RT-PCR. JeKo-1 MCL cell line was found to be the most sensitive to rGel/BLyS (targeting index=100,000). Among MCL cell lines, Granta 519 cells were most resistant to rGel/BLyS and rGel itself. The inventors were unable to detect a direct correlation between the expression levels of BLyS receptor(s) and sensitivity to rGel/BLyS and examined different cytotoxic mechanisms of rGel/BLyS in these two MCL cell lines to identify mechanisms which may account for the divergent cytotoxic effects. The inventors observed that rGel/BLyS can rapidly internalize into most sensitive cell line, JeKo-1, but not Granta 519 (Data not shown). This result indicates that rGel/BLyS can internalize into JeKo-1 cells after binding to BLyS receptors and deliver the rGel toxin to JeKo-1 MCL cell line expressing three BLyS receptors.

Mantle cell lymphoma (MCL) is a distinct type of B-cell non-Hodgkin lymphoma that is characterized by a constellation of morphologic, immunophenotypic, and cytogenetic features and overexpresses cyclin D1. Conventional cytotoxic therapy is not effective and the overall prognosis is poor (Leonard et al., 2001). MCL remains the most therapeutically resistant of the B cell non-Hodgkin's lymphoma (Weisenburger et al., 2000). The resistance of MCL to current chemotherapy regime indicates that new therapeutic approachs to MCL are clearly needed. The results indicate that the fusion toxin rGel/BLyS is an excellent therapeutic agent at least for mantle cell lymphoma, a lymphoma that is refractory to most current chemotherapy regimes.

Multiple myeloma (MM) is a B-cell neoplasia that is characterized by clonal expansion of plasma cells in the bone marrow and remains incurable despite conventional, high-dose therapies. Greenstein et al. (2003) established three MM1.S (dexamethasone-sensitive cells), MM1RE (early form of dexamethasone-resistant MM1.R cells), and MM1.RL (late form of dexamethasone-resistant MM1.R cells) to study the etiology of MM, effects of chemotherapeutic agents, and development of clinical resistance. Interestingly, the inventors found that dexamethasone-sensitive (MM1.S) and -resistant (MM1.R) cell lines were equally sensitive to rGel/BLyS (IC₅₀ values of 300 nM for rGel/BLyS). The inventors also found that parental melphalan-sensitive RPMI8226 and -resistant 8226/LR-5 multiple myeloma cells were equally sensitive to rGel (200 versus 280, respectively) but not to rGel/BLyS (10 versus 110, respectively) (Table 4). This indicates that cellular resistance to dexamethasone does not appear to result in development of cross-resistance to the fusion toxin rGel/BLyS whereas cellular resistance to melphalan does appear to result in development of cross-resistance to melphalan. Signaling studies are ongoing to understand the cytotoxic mechanism of rGel/BLyS in both drug-sensitive and -resistant cell lines.

Taken together, the data indicate that rGel/BLyS is a good candidate for the treatment of at least mantle cell lymphoma and, in specific embodiments, BLyS serves as a targeting ligand for the specific delivery of toxin to B-cells expressing one or more of the receptors for BLyS.

### Example 13

### Exemplary Materials and Methods

The present example provides exemplary materials and methods for use in the present invention.

Materials

The PCR reagents, RNA isolation kit, and reverse transcription (RT)-PCR kits were all obtained from Life Technologies, Inc. (Frederick, Md.). The restriction enzymes were purchased from New England Biolabs (Beverly, Mass.). RNA and DNA purification kits were obtained from Qiagen, Inc. (Valencia, Calif.). Bacterial strains, pET bacterial expression plasmids, and recombinant enterokinase (rEK) were obtained from Novagen (Madison, Wis.). Hi-Trap chelating HP resin and other chromatography resins were purchased from Amersham Bioscience (Uppsala, Sweden). Mouse monoclonal anti-PARP antibody (Ab), rabbit anti-cyclin D1 Ab, and goat anti-β-actin Ab were obtained from Santa Cruz Biotechnology (Santa Cruz, Calif.). Rabbit polyclonal anti-active caspase-3 Ab was purchased from BD Biosciences (San Jose, Calif.).

Cell Lines and Cell Culture

The multiple myeloma doxorubicin-sensitive cell line, MM1.S, and -resistant cell line, MM1.R, were kindly provided by Dr. Varsha Gandhi (M. D. Anderson Cancer Center, Houston, Tex.). The plasmacytoma myeloma melphalan resistant cell line, 8226/LR-5, was kindly provided by Dr. William Dalton (Arizona Cancer Center, Tucson) (Phillips et al., 2003). The four mantle cell lymphoma (MCL) cell lines (JeKo-1, SP53, Mino, and Granta-519) were kindly provided by Dr. Hesham Amin (M. D. Anderson Cancer Center, Houston, Tex.) (Kanakaraj et al., 2001). Jurkat, KBM-5, THP-1, HL-60, IM-9, RPMI 8226, MM1.S, MM1.R, and JeK-1 cell lines were grown in RPMI 1640 medium (ATCC, Manassas, Va.) supplemented with 10% heat-inactivated fetal bovine serum (FBS), 100 units/ml penicillin and 100 μg/ml streptomycin. 5 μM melphalan was included in the RPMI 1640 medium (ATCC) for RPMI8226/LR-5 cell line. Granta 519 cell line was grown in DMEM (Invitrogen, Grand Island, N.Y.) supplemented with 10% heat-inactivated fetal bovine serum (FBS), 100 units/ml penicillin and 100 μg/ml streptomycin. SP53 and Mino cell lines were grown in RPMI 1640 medium (ATCC) supplemented with 20% heat-inactivated FBS, 100 units/ml penicillin and 100 μg/ml streptomycin.

Construction of the rGel/BLyS Fusion Toxin

RNA from JeKo-1 cells was isolated and the cDNA encoding human BLyS was amplified by RT-PCR using the following primers: BLySf (5′→3′): GGAGAAGGCAACTCCAGTCAGAAC (SEQ ID NO:22) and BlySr (5′→3′): GTCCATGTCTTTGGGGATGAATTG (SEQ ID NO:23) (Schneider et al., 1999). A rGel/BLyS fusion DNA construct was generated by the splice overlap extension PCR (OE-PCR) (Weisenburger et al., 2000) method using the entire coding region of the BLyS and recombinant gelonin as DNA templates to amplify individual gene fragments. To construct the rGel/BLyS fusion DNA construct, an upstream OE-PCR fragment encoding the enterokinase and the restriction enzymes Kpn I digestion sites was amplified from the N-terminal portion of the the recombinant gelonin using the oligonuceotide primers PETgelfor, (5′-AGCCCAGATCTGGGTACCGACGACGACGACAAGGGCCTGGACACCGTGAGC-3′; SEQ ID NO:16); rGelbaclink, (5′-GCTCCCGCCTCCCCCTTTAGGATCTTT-3′; SEQ ID NO:17). An adjoining downstream OE-PCR fragment encoding a G4S linker and a restriction enzyme Xho I site was amplified from the BLyS gene using the oligonucleotide primers BLySfor, (5′-GGGGGAGGCGGGAGCGCCGTTCAGGGTCCA-3′; SEQ ID NO:18), and BLySrev, (5′-GCCGTCGACCTCGAGTCATTACAGCAGTTTCAATGC-3′; SEQ ID NO:19). The upstream and downstream OE-PCR fragments were then reassembled as a full-length rGel/BLyS fusion gene by an additional PCR step using a pair of oligonucleotide primers PETgelfor and BLySrev flanking the 5′- and 3′-end (see above). The final PCR fragment was purified and cleaved with Kpn I and Xho I restriction endonucleases and then cloned into the expression vector pET-32a (Novagen) that utilizes the T7 promoter for the transcriptional control of the inserted fusion gene. The fusion toxin rGel/BLyS gene constructs were verified by DNA sequencing and correct fusion constructs were transformed into Escherichia coli strain AD494 (DE3) for protein expression of the fusion toxin.

Expression and Induction of rGel/BLyS in E. coli

Bacterial colonies transformed with the plasmid carrying the rGel/BLyS insert were cultured in Luria broth medium containing 400 μg/ml ampicillin and 30 μg/ml kanamycin at 37° C. overnight in a shaker incubator at 235 rpm. The bacterial cultures were then diluted 1:100 with fresh LB medium containing antibiotics and grown to A₆₀₀=0.8 at 37° C. Thereafter, the cultures were diluted 1:1 with fresh LB medium plus 400 μg/ml of ampicillin and 30 μg/ml of kanamycin and expression of the growth factor fusion toxin rGel/BLyS was induced at 23° C. by addition of 100 μM isopropyl-1-thio-β-D-galactopyranoside (IPTG) overnight. The cells were collected by centrifugation, resuspended in 40 mM Tris-HCl (pH 8), and frozen (−80° C.).

Purification of rGel/BLyS

Frozen bacterial cells were thawed and then lysed by physical disruption (Bead Beater, Biospec Products, Bartlesville, Okla.) at 4° C. The bacterial lysates were ultracentrifuged at 40,000×g for 1.5 hr. The final concentration of NaCl in supernatant was adjusted to 500 mM NaCl and then loaded onto Hi-Trap chelating HP resin (Amersham) charged with 200 mM Ni₂SO₄. The column was washed with 40 mM Tris-HCl (pH 8) and 500 mM NaCl containing 30 mM imidazole and eluted with 40 mM Tris-HCl (pH 8) and 500 mM NaCl containing 300 mM imidazole. The rGel/BLyS containing fractions were pooled and dialyzed into dialysis buffer containing 20 mM Tris-HCl (pH7.4) and 150 mM NaCl. To remove the histidine tag, the rGel/BLyS fusion toxin containing histidine tag was digested overnight at room temperature with recombinant enterokinase (rEK, Novagen). The non-specific protein and the 20 kDa histidine tag were removed by ion exchange chromatography through Q-Sepharose Fast Flow (Amersham) and by affinity chromatography through Blue-Sepharose CL-6B (Amersham). The purified rGel/BLyS samples were filter-sterilized, aliquoted, and stored at 4° C.

Analysis of rGel/BLyS

To assess the presence of rGel toxin and BLyS components in the rGel/BLyS fusion toxin, the final samples were analyzed by 12% SDS-PAGE under reducing conditions. Purified rGel/BLyS (5 μg) were separated by SDS-PAGE (8-15%) and electrophoretically transferred to PVDF membranes (Millipore Corporation, Bedford, Mass.) overnight at 4° C. in transfer buffer [25 mM Tris-HCl (pH 8.3), 190 mM Glycine, 20% methanol]. The PVDF membranes were blocked for 1 hour in Tris-buffered saline (TBS) containing 5% non-fat milk and then probed with rabbit anti-gelonin Ab or goat anti-BLyS Ab. Goat anti-rabbit or swain anti-goat antibodies conjugated with horseradish peroxidase (Bio-Rad Laboratories, Hercules, Calif.) were used to visualize immunoreactive proteins at a 1:4000 dilution using ECL detection reagent (Amersham Pharmacia Biotech Inc., Piscataway, N.J.). BLyS or recombinant gelonin were used as positive controls. Data are presented as the relative density of protein bands normalized to β-actin. The intensity of the bands was quantified using Histogram.

Cell-Free Protein Synthesis Inhibitory Activity of rGel/BLyS

The n-glycosidic inhibitory activity of the recombinant rGel toxin was assessed compared to that of the rGel/BLyS fusion toxin. The toxin-induced inhibition of protein production was assessed using a rabbit reticulocyte lysate assay as specified by the manufacturer (Promega, Madison, Wis.) and as described previously (Hale, 2001).

BLyS Receptor Binding Activity of rGel/BLyS

To assess the BLyS receptor binding activity of the fusion toxin rGel/BLyS, JeKo-1 and HL-60 cells were immobilized onto poly-L-lysine-coated 96 well plates (Becton Dickinson Labware, Franklin Lakes, N.J.) at a density of 1×10⁵ cells/well. The plates were rehydrated, blocked with 3% BSA, and then incubated with different concentrations of rGel/BLyS or rGel in dilution buffer (PBS, 0.1% Tween 20, 0.1% BSA) for 2 hours at room temperature. After washing, plates were incubated with rabbit anti-gelonin polyclonal antibody for 1 hour at room temperature, washed 4 times with PBST (1 μg/ml in PBS, 0.1% Tween 20, 0.1% BSA), and then 100 μl of peroxidase-conjugated goat anti-rabbit IgG (1 μg/ml in dilution buffer; Vector, Burlingame, Calif.) was added to each well. Plates were incubated for 1 hour at room temperature, washed 4 times with PBST, and developed with tetramethylbenzidine substrate (Sigma). Absorption at 450 nm was measured with Spentra Max 3000 instrument (Molecular Devices, Sunnyvale, Calif.)

Detection of BLyS, BAFF-R, TACI, and BCMA Expression on Various Cell Lines

The expression of BLyS, BAFF-R, TACI, and BCMA was assessed in 13 cell lines by reverse transcription-polymerase chain reaction (RT-PCR) analysis. Total isolated RNA from 13 cell lines was used to synthesize the first-strand cDNA that in turn was amplified by PCR by using specific primers designed to amplify human BLyS (313 bp) (Schneider et al., 1999), BAFF-R (256 bp) (Kern et al., 2004), TACI (196 bp) (Phillips et al., 2003), and BCMA (285 bp) (Phillips et al., 2003). GADPH was used as a control.

Cytotoxic Activity of rGel/BLyS and Competitive Inhibition by Free BLyS or Decoy Receptors

To examine the comparative IC50 values of rGel/BLyS against 13 cell lines, thirteen cell lines were seeded (5×10³ cells/well) in flat-bottom 96-well microtiter plates (Becton Dickinson) and rGel itself or rGel/BLyS were added in quadruplicate wells. After 96 hr, 50 μl of XTT labeling mixture (Roche) was added to each well, after which the cells incubated for overnight. The spectrophotometric absorbance was measured at 450 nm using an ELISA reader (Bio-Tek Instruments, Inc., Winooski, Vt.).

To assess the specificity of rGel/BLyS against BLyS receptor expressing cells, we treated JeKo-1 cells with BLyS itself, free rGel, CTP/rGel (non-B cell targeting chemical conjugate), rGel/BLyS, or medium were added in quadruplicate wells. For competitive inhibition assays, JeKo-1 cells were seeded (5×10³/well) in flat-bottom 96-well microtiter plates (Becton Dickinson) and pre-treated with 1 nM of BLyS, 50 nM of BLyS, 10 μg/ml of BAFF-R:Fc, 10 μg/ml of TACI:Fc, or 10 μg/ml of BCMA:Fc for 2 hr, and then rGel/BLyS or rGel itself was added in quadruplicate wells.

Internalization of rGel/BLyS into JeKo-1 Mantle Cell Lymphoma (MCL) Cell Line

The JeKo-1 MCL cell line was added to polylysine-coated 16 well chamber slides (Nunc, Rochester, N.Y.) at 1×10⁴ cells/well and treated with 100 nM rGel or 100 nM rGel//BLyS at various times. Cells were then placed onto slides using cytospin (Shandon, Pittsburgh, Pa.), and then proteins bound to the cell surface were stripped by 10 min incubation with glycine buffer [500 mM NaCl and 0.1 M glycine (pH 2.5)], neutralized for 5 min with 0.5 M Tris (pH 7.4), washed briefly with PBS, and then fixed in 3.7% formaldehyde (Sigma, St. Louis, Mo.) for 20 min at room temperature, followed by a brief rinse with PBS. Cells were then permeabilized for 10 min in PBS containing 0.2% Triton X-100, washed three times with PBS, and blocked with PBS containing 3% BSA for 1 hr at room temperature. After a brief wash with PBS, cells were incubated with rabbit anti-rGel polyclonal antibody diluted 1:500 in PBS containing 0.1% Tween 20 and 0.2% BSA for 1 hr at room temperature. Cells were washed three times in PBS containing 0.1% Tween 20 for 15 min and incubated with a 1:100 dilution of FITC-coupled anti-rabbit IgG (Sigma) containing 1 μg/ml of propidium iodide (PI). After three washes with PBS containing 0.1% Tween 20, cells were washed once in PBS for 10 min and mounted in DABCO mounting medium. Slides were then analyzed with a Zeiss LSM510 laser scanning microscope (Carl Zeiss, Jena, Germany).

Detection of Apoptosis

Apoptosis was detected by the TdT-mediated dUTP nick end labeling (TUNEL) assay. To assess apoptosis, JeKo-1 cells were added to polylysine-coated 16 well chamber slides (Nunc) at 5×10³ cells/well and treated with 100 pM BLyS, 100 pM rGel, 100 pM rGel/BLyS or media for 4 days. Floating cells were then collected and affixed to slides using cytospin (Shandon), dried and then fixed in 3.7% formaldehyde (Sigma) for 20 min at room temperature and followed by a brief rinse with PBS. Cells were then permeabilized for 10 min in PBS containing 0.2% Triton X-100 and 0.1% sodium citrate and washed three times with PBS, and blocked with PBS containing 3% BSA for 1 hr at room temperature. Fixed cells were stained with in situ cell death detection kit (Roche). After a final wash step, the slides were mounted in mounting medium and analyzed under fluorescence microscope.

Western Blot Analysis

To examine the effects of rGel/BLyS on the expression of cleaved caspase-3 and PARP, JeKo-1 cells were seeded at 5×10⁵ cells/24-well plate, and then treated with 100 pM of BLyS, 100 pM rGel, 100 pM rGel/BLyS or media. After 96 hr, cells were washed twice with phosphate buffered saline (PBS) and lysed on ice for 20 min in 0.3 ml of lysis buffer (10 mM Tris-HCl, pH 8, 60 mM KC1, 1 mM EDTA, 1 mM DTT, 0.2% NP-40). Cell lysates (50 μg) were separated by SDS-PAGE (8-15%) and electrophoretically transferred to PVDF membranes (Millipore Corporation, Bedford, Mass.) overnight at 4° C. in transfer buffer [25 mM Tris-HCl (pH 8.3), 190 mM Glycine, 20% methanol]. The PVDF membranes were blocked for 1 hour in Tris-buffered saline (TBS) containing 5% non-fat milk and then probed with mouse monoclonal anti-PARP antibody (Ab), rabbit polyclonal anti-active caspase-3 Ab, or goat anti-β-actin. Goat anti-mouse/anti-rabbit or swain anti-goat antibodies conjugated with horseradish peroxidase (Bio-Rad Laboratories, Hercules, Calif.) were used to visualize immunoreactive proteins at a 1:4000 dilution using ECL detection reagent (Amersham Pharmacia Biotech Inc., Piscataway, N.J.). Data are presented as the relative density of protein bands normalized to β-actin. The intensity of the bands was quantified using Histogram.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### Patents and Patent Applications

U.S. Pat. No. 5,631,348

U.S. Pat. No. 6,669,938

U.S. Pat. No. RE37,462

U.S. Pat. No. 6,750,329

U.S. Pat. No. 6,599,505

U.S. Pat. No. 6,214,974

U.S. Pat. No. 5,624,827

U.S. Pat. No. 5,053,226

### PUBLICATIONS

Amin H M, McDonnell T J, Medeiros J, et al. Characterization of 4 mantle cell lymphoma cell lines. Arch Pathol Lab Med. 2003; 127:424-431.
Backer M V, Backer J M. Targeting endothelial cells overexpressing VEGFR-2: selective toxicity of Shiga-like toxin-VEGF fusion proteins. Bioconjug Chem. 2001; 12:1066-1073.
Bellamy W T, Dalton W S, Gleason M C, Grogan T M, Trent J M. Development and characterization of a melphalan-resistant human multiple myeloma cell line. Cancer Res. 1991; 51 :995-1002.
Briones J, Timmerman J M, Hilbert D M, Levy R. BLyS and BLyS receptor expression in non-Hodgkin's lymphoma. Exp Hematol. 2002; 30:135-141.
Chen, G., Zou, M. J., Peng, S., Wang, J. X. Cloning, expression and activity determination of the recombinant human soluble B lymphocyte stimulator and its two mutants. Sheng Wu Hua Xue Yu Sheng Wu Wu Li Xue Bao (Shanghai). 2002; 34(6):731-6.
Chen, G., Peng, S., Zou, M., Xu, H., Xu, D., Wang, J. Construction and function of two Cys146-mutants with high activity, derived from recombinant human soluble B lymphocyte stimulator. 2004; 136(1):73-9.
Chen, G., Du, H., Zhang, Z., Peng, S., Xu, D., Wang, J. Primary immune effects of eukaryotic expression plasmids encoding two hyperactive mutants of human soluble B lymphocyte stimulator. J. Clin. Immunol. 25(5):445-51.
Craxton A, Magaletti D, Ryan E J, Clark E A. Macrophage- and dendritic cell-dependent regulation of human B-cell proliferation requires the TNF family ligand BAFF. Blood. 2003; 101:4464-4471.
Do R K, Chen-Kiang S. Mechanism of BLyS action in B cell immunity. Cytokine Growth Factor Rev. 2002; 13:19-25.
Duzkale H, Pagliaro L C, Rosenblum M G, et al. Bone marrow purging studies in acute myelogenous leukemia using the recombinant anti-CD33 immunotoxin HuM195/rGel. Biol Blood Marrow Transplant. 2003; 9:364-372.
Foss F M. Interleukin-2 fusion toxin: targeted therapy for cutaneous T cell lymphoma. Ann NY Acad. Sci. 2001; 941:166-176.
Frankel A, Tagge E, Chandler J, et al. IL2-ricin fusion toxin is selectively cytotoxic in vitro to IL2 receptor-bearing tumor cells. Bioconjug Chem. 1995; 6:666-672.
Greenstein S, Krett N L, Kurosawa Y, et al. Characterization of the MM1.1 human multiple myeloma (MM) cell lines: A model system to elucidate the characteristics, behavior, and signaling of steroid-sensitive and -resistant MM cells. Exp Hematol 2003; 31: 271-282.
Hahne M, Kataoka T, Schroter M, et al. APRIL, a new ligand of the tumor necrosis factor family, stimulates tumor cell growth. J Exp Med. 1998; 188:1185-1190.
Hale M L. Microtiter-based assay for evaluating the biological activity of ribosome-inactivating proteins. Pharmacol Toxicol. 2001; 88:255-260.
Ho S N, Hunt H D, Horton R M, Pullen J K, Pease L R. Site-directed mutagenesis by overlap extension using the polymerase chain reaction. Gene. 1989; 77:51-59.
Hotz H G, Gill P S, Masood R, et al. Specific targeting of tumor vasculature by diphtheria toxin-vascular endothelial growth factor fusion protein reduces angiogenesis and growth of pancreatic cancer. J Gastrointest Surg. 2002; 6:159-166.
Joshi B H, Kawakami K, Leland P, Puri R K. Heterogeneity in interleukin-13 receptor expression and subunit structure in squamous cell carcinoma of head and neck: differential sensitivity to chimeric fusion proteins comprised of interleukin-13 and a mutated form of Pseudomonas exotoxin. Clin Cancer Res. 2002; 8:1948-1956.
Kanakaraj P, Migone T-S, Nardelli B, et al. BLyS binds to B cells with high affinity and induces activation of the transcription factors NF-B and ELF-1. Cytokine. 2001; 13:25-31.
Kern C, Cornuel J F, Billard C, et al. Involvement of BAFF and APRIL in the resistance to apoptosis of B-CLL through an antocrine pathway. Blood. 2004; 103:679-688.
Kiyokawa T, Williams D P, Snider C E, Strom T B, Murphy J R. Protein engineering of diphtheria-toxin-related interleukin-2 fusion toxins to increase cytotoxic potency for high-affinity IL-2-receptor-bearing target cells. Protein Eng. 1991; 4:463-468.
Kreitman R J, Pastan I. Immunobiological treatments of hairy-cell leukaemia. Best Pract Res Clin Haematol. 2003; 16:117-133.
Laabi Y, Gras M P, Carbonnel F, et al. A new gene, BCM, on chromosome 16 is fused to the interleukin 2 gene by a t(4;16)(q26;p13) translocation in a malignant T cell lymphoma. EMBO J. 1992; 11:3897-3904.
Lakkis F, Landgraf B, Wen Z, Strom T B, Murphy J R. Phe496 and Leu497 are essential for receptor binding and cytotoxic action of the murine interleukin-4 receptor targeted fusion toxin DAB389-mIL-4. Protein Eng. 1992; 5:241-248.
Leonard J P, Schattner E J, Coleman M. Biology and management of mantle cell lymphoma. Curr Opin Oncol. 2001; 13:342-347.
Liger D, vanderSpek J C, Gaillard C, et al. Characterization and receptor specific toxicity of two diphtheria toxin-related interleukin-3 fusion proteins DAB389-mIL-3 and DAB389-(Gly4Ser)₂-mIL-3. FEBS Lett. 1997; 406:157-161.
Liu Y, Cheung L H, Thorpe P, Rosenblum M G. Mechanistic studies of a novel human fusion toxin composed of vascular endothelial growth factor (VEGF)121 and the serine protease granzyme B: directed apoptotic events in vascular endothelial cells. Mol Cancer Ther. 2003; 2:949-959.
Mackay F, Ambrose C. The TNF family members BAFF and APRIL: the growing complexity. Cytokine Growth Factor Rev. 2003; 14:311-324.
Mackay F, Schneider P, Rennert P, Browning J. BAFF and APRIL: a tutorial on B cell survival. Annu Rev Immunol. 2003; 21:231-264.
May R D, Vitetta E S, Moldenhauer G, Dorken B. Selective killing of normal and neoplastic human B cells with anti-CD19- and anti-CD22-ricin A chain immunotoxins. Cancer Drug Deliv. 1986; 3:261-272.
Moore P A, Belvedere O, Orr A, et al. BLyS: member of the tumor necrosis factor family and B lymphocyte stimulator. 1999; 285:260-263.
Nardelli B, Belvedere O, Roschke V, et al. Synthesis and release of B-lymphocyte stimulator from myeloid cells. Blood. 2001; 97:198-204.
Nardelli B, Moore P A, Li Y, Hilbert D M. B lymphocyte stimulator (BLyS): a therapeutic trichotomy for the treatment of B lymphocyte diseases. Leuk Lymphoma. 2002; 43:1367-1373.
Novak A J, Bram R J, Kay N E, Jelinek D F. Aberrant expression of B-lymphocyte stimulator by B chronic leukemia cells: a mechanism for survival. Blood. 2002; 100:2973-2979.
O'Boyle K P, Colletti D, Mazurek C, et al. Potentiation of antiproliferative effects of monoclonal antibody Lym-1 and immunoconjugate Lym-1-gelonin on human Burkitt's lymphoma cells with gamma-interferon and tumor necrosis factor. J Immunother Emphasis Tumor Immunol. 1995; 18:221-230.
Phillips T A, Ni J, Hunt J S. Cell-specific expression of B lymphocyte (APRIL, BLyS)- and Th2 (CD30L/CD153)-promoting tumor necrosis factor superfamily ligands in human placentas. J Leukoc Biol. 2003; 74:81-87.
Riccobene T A, Miceli R C, Lincoln C, et al. Rapid and specific targeting of ¹²⁵I-labeled B lymphocyte stimulator to lymphoid tissues and B cell tumors in mice. J Nucl Med. 2003; 44:422-433.
Rosenblum M G, Cheung L H, Liu Y, Marks J W. Design, expression, purification, and characterization, in vitro and in vivo, of an antimelanoma single-chain Fv antibody fused to the toxin gelonin. Cancer Res. 2003; 63:3995-4002.
Rosenblum M G, Murray J L, Cheung L, Rifkin R, Salmon S, Bartholomew R. A specific and potent immunotoxin composed of antibody ZME-018 and the plant toxin gelonin. Mol Biother. 1991; 3:6-13.
Rosenblum M G, Shawver L K, Marks J W, Brink J, Cheung L, Langton-Webster B. Recombinant immunotoxins directed against the c-erb-2/HER2/neu oncogene product: in vitro cytotoxicity, pharmacokinetics, and in vivo efficacy studies in xenograft models. Clin Cancer Res. 1999; 5:865-874.
Rosenblum M G, Zuckerman J E, Marks J W, Rotbein J, Allen W R. A gelonin-containing immunotoxin directed against human breast carcinoma. Mol Biother. 1992; 4:122-129.
Scapini P, Nardelli B, Nadali G, et al. G-CSF-stimulated neurophils are a prominent source of functional BLyS. J Exp Med. 2003; 197:297-302.
Schneider P, Mackay F, Steiner V, et al. BAFF, a novel ligand of the tumor necrosis factor family, stimulates B cell growth. 1999; 189:1747-1756.
Schnell R, Vitetta E, Schindler J, et al. Treatment of refractory Hodgkin's lymphoma patients with an anti-CD25 ricin A-chain immunotoxin. Leukemia. 2000; 14:129-135.
Shapiro M E, Kirkman R L, Kelley V R, Bacha P, Nichols J C, Strom T B. In vivo studies with chimeric toxins. Interleukin-2 fusion toxins as immunosuppressive agents. Targeted Diagn Ther. 1992; 7:383-393.
Thompson J S, Bixler S A, Qian F, et al. BAFF-R, a newly identified TNF receptor that specifically interacts with BAFF. Science. 2001; 293:2108-2111.
Uckun F M, Jaszcz W, Ambrus J L, et al. Detailed studies on expression and function of CD19 surface determinant by using B43 monoclonal antibody and the clinical potential of anti-CD19 immunotoxins. Blood. 1988; 71:13-29.
van Horssen P J, van Oosterhout Y V, Evers S, et al. Influence of cytotoxicity enhancers in combination with human serum on the activity of CD22-recombinant ricin A against B cell lines, chronic and acute lymphocytic leukemia cells. Leukemia. 1999; 13:241-249.
van Oosterhout Y V, van den Herik-Oudijk I E, Wessels H M, de Witte T, van de Winkel J G, Preijers F W. Effect of isotype on internalization and cytotoxicity of CD19-ricin A immunotoxins. Cancer Res. 1994; 54:3527-3532.
Veenendaal L M, Jin H, Ran S, et al. In vitro and in vivo studies of a VEGF121/rGelonin chimeric fusion toxin targeting the neovasculature of solid tumors. Proc Natl Acad Sci USA. 2002; 99:7866-7871.
von Bulow G U, Bram R J. NFAT activation induced by a CAML-interacting member of the tumor necrosis factor receptor superfamily. Science. 1997; 278:138-141.
Weisenburger D D, Vose J M, Greiner T C, et al. Mantle cell lymphoma: a clinicopathologic study of 68 cases from the Nebraska Lymphoma Study Group. Am J Hematol. 2000; 64:190-196.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim:

1. A composition comprising a BLyS polypeptide conjugated to a therapeutic agent.

2. The composition of claim 1, wherein the therapeutic agent is further defined as one or more of a cytotoxic agent, a chemotherapeutic agent, an antibody, a cytokine, a pro-apoptotic agent, or an angiogenic inhibitor.

3. The composition of claim 2, wherein the cytotoxic agent comprises a peptide, a polypeptide, or a small molecule.

4. The composition of claim 1, wherein the composition is further defined as a fusion protein.

5. The composition of claim 1, wherein the BLyS polypeptide comprises a B-cell targeting domain.

6. The composition of claim 1, wherein the BLyS polypeptide comprises a D-E receptor recognition loop.

7. The composition of claim 2, wherein the BLyS polypeptide and the cytotoxic agent are chemically conjugated.

8. The composition of claim 2, wherein the cytotoxic agent comprises a gelonin molecule.

9. The composition of claim 8, wherein the gelonin molecule is 5′ to the BLyS polypeptide.

10. The composition of claim 2, wherein the cytotoxic agent is selected from the group consisting of ricin A, diphtheria toxin, abrin, dodecandrin, tricosanthin, tricokirin, bryodin, mirabilis antiviral protein, barley ribosome-inactivating protein (BRIP), pokeweed antiviral protein (PAPs), saporin, luffin, Pseudomonas exotoxin, and momordin.

11. The composition of claim 1, wherein the composition comprises a recombinant polypeptide.

12. The composition of claim 1, further defined as being comprised in a pharmaceutically acceptable carrier.

13. A host cell comprising the composition of claim 1.

14. A method of treating an individual with a B-cell proliferative disorder comprising administering to the individual a therapeutically effective amount of a composition of claim 1.

15. The method of claim 14, wherein the B-cell proliferative disorder is selected from the group consisting of: B-cell chronic Lymphocytic leukemia/small lymphocytic lymphoma B-cell prolymphocytic leukemia, Immunocytoma/lymphoplasmacytic lymphoma (+/−Waldenstrom's macroglobulinemia), Mantle cell lymphoma, Marginal Zone B-cell Lymphoma of mucosa-associated lymphoid tissue (MALT) type, Splenic marginal zone B-cell Lymphoma (+/−villous Lymphocytes), Hairy cell leukemia, Diffuse large B-cell Lymphoma, Mediastinal (Thymic) large B-cell Lymphoma, Intravascular large B-cell Lymphoma, Burkitt Lymphoma, Plasma cell myeloma (multiple myeloma), Monoclonal gammopathy of undetermined significance (MGUS), Indolent myeloma, Smoldering myeloma, Osteosclerotic myeloma (POEMS syndrome), Plasma cell leukemia, Non-secretory myeloma, Plasmacytomas, Solitary plasmacytoma of bone, Extramedullary plasmacytoma, Waldenstrom's macroglobulinemia, Heavy Chain Disease (HCD), Immunoglobulin deposition diseases, Systemic light chain disease, Primary amyloidosis, Hodgkins Disease, Non-Hodgkins Disease, Lupus, or arthritis.

16. A method of selectively targeting a cell expressing a BLyS receptor comprising contacting the cell with an effective amount of a composition of claim 1.

17. A method of monitoring therapy in an individual with B-cell proliferative disorder, comprising administering to the individual a therapeutically effective amount of a composition of claim 1.

18. A kit comprising the composition of claim 1 housed in a suitable container.

19. The kit of claim 18, wherein the composition is comprised in a pharmaceutically acceptable carrier.

20. The kit of claim 18, wherein the composition is suitably aliquoted for delivery to an individual.


SEQUENCE LISTING (23 sequences)

SEQ ID NO: 1 - protein, 285 aa, HUMAN
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 2 - protein, 309 aa, MOUSE
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 3 - DNA, 1710 bp, Mouse
ggcacgaggcagattgagcaatccatggaaggccagagccagagaacctacttcagggtagcaaaagatgcagaagaaagtcaggagagcgctcctgggggaacccagccctgccatgctctgagggcagtctcccaggacacagatgacaggaaatgacccacccctgtggtcacttactccaaaggcctagaccttcaaagtgctcctcgtggaatggatgagtctgcaaagaccctgccaccaccgtgcctctgtttttgctccgagaaaggagaagatatgaaagtgggatatgatcccatcactccgcagaaggaggagggtgcctggtttgggatctgcagggatggaaggctgctggctgctaccctcctgctggccctgttgtccagcagtttcacagcgatgtccttgtaccagttggctgccttgcaagcagacctgatgaacctgcgcatggagctgcagagctaccgaggttcagcaacaccagccgccgcgggtgctccagagttgaccgctggagtcaaactcctgacaccggcagctcctcgaccccacaactccagccgcggccacaggaacagacgcgctttccagggaccagaggaaacagaacaagatgtagacctctcagctcctcctgcaccatgcctgcctggatgccgccattctcaacatgatgataatggaatgaacctcagaaacatcattcaagactgtctgcagctgattgcagacagcgacacgccgactatacgaaaaggaacttacacatttgttccatggcttctcagctttaaaagaggaaatgccttggaggagaaagagaacaaaatagtggtgaggcaaacaggctatttcttcatctacagccaggttctatacacggaccccatctttgctatgggtcatgtcatccagaggaagaaagtacacgtctttggggacgagctgagcctggtgaccctgttccgatgtattcagaatatgcccaaaacactgcccaacaattcctgctactcggctggcatcgcgaggctggaagaaggagatgagattcagcttgcaattcctcgggagaatgcacagatttcacgcaacggagacgacaccttctttggtgccctaaaactgctgtaactcacttgctggagtgcgtgatccccttccctcgtcttctctgtacctccgagggagaaacagacgactggaaaaactaaaagatggggaaagccgtcagcgaaagttttctcgtgacccgttgaatctgatccaaaccaggaaatataacagacagccacaaccgaagtgtgccatgtgagttatgagaaacggagcccgcgctcagaaagaccggatgaggaagaccgttttctccagtcctttgccaacacgcaccgcaaccttgctttttgccttgggtgacacatgttcagaatgcagggagatttccttgttttgcgatttgccatgagaagagggcccacaactgcaggtcactgaagcattcacgctaagtctcaggatttactctcccttctcatgctaagtacacacacgctcttttccaggtaatactatgggatactatggaaaggttgtttgtttttaaatctagaagtcttgaactggcaatagacaaaaatccttataaattcaagtgtaaaataaacttaattaaaaaggtttaagtgtgaaaaaaaa

SEQ ID NO: 4 - protein, 293 aa, HUMAN
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 5 - protein, 184 aa, HUMAN
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 6 - protein, 184 aa, HUMAN
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 7 - protein, 251 aa, gelonin plant
GLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPK

SEQ ID NO: 8 - protein, 408 aa, ARTIFICIAL SEQUENCE
  Conjugated Polypeptide
AVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLLGGGGSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPK

SEQ ID NO: 9 - DNA, 1224 bp, ARTIFICIAL SEQUENCE
  Polynucleotide encoding the conjugated polypeptide
gccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctggggggaggcgggagcggcctggacaccgtgagctttagcactaaaggtgccacttatattacctacgtgaatttcttgaatgagctacgagttaaattgaaacccgaaggtaacagccatggaatcccattgctgcgcaaaaaatgtgatgatcctggaaagtgtttcgttttggtagcgctttcaaatgacaatggacagttggcggaaatagctatagatgttacaagtgtttatgtggtgggctatcaagtaagaaacagatcttacttctttaaagatgctccagatgctgcttacgaaggcctcttcaaaaacacaattaaaacaagacttcattttggcggcagctatccctcgctggaaggtgagaaggcatatagagagacaacagacttgggcattgaaccattaaggattggcatcaagaaacttgatgaaaatgcgatagacaattataaaccaacggagatagctagttctctattggttgttattcaaatggtgtctgaagcagctcgattcacctttattgagaaccaaattagaaataactttcaacagagaattcgcccggcgaataatacaatcagccttgagaataaatggggtaaactctcgttccagatccggacatcaggtgcaaatggaatgttttcggaggcagttgaattggaacgtgcaaatggcaaaaaatactatgtcaccgcagttgatcaagtaaaacccaaaatagcactcttgaagttcgtcgataaagatcctaaa

SEQ ID NO: 10 - protein, 408 aa, ARTIFICIAL SEQUENCE
  Conjugated Polypeptide
GLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKGGGGSAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 11 - DNA, 1224 bp, ARTIFICIAL SEQUENCE
  Polynucleotide encoding the conjugated polypeptide
ggcctggacaccgtgagctttagcactaaaggtgccacttatattacctacgtgaatttcttgaatgagctacgagttaaattgaaacccgaaggtaacagccatggaatcccattgctgcgcaaaaaatgtgatgatcctggaaagtgtttcgttttggtagcgctttcaaatgacaatggacagttggcggaaatagctatagatgttacaagtgtttatgtggtgggctatcaagtaagaaacagatcttacttctttaaagatgctccagatgctgcttacgaaggcctcttcaaaaacacaattaaaacaagacttcattttggcggcagctatccctcgctggaaggtgagaaggcatatagagagacaacagacttgggcattgaaccattaaggattggcatcaagaaacttgatgaaaatgcgatagacaattataaaccaacggagatagctagttctctattggttgttattcaaatggtgtctgaagcagctcgattcacctttattgagaaccaaattagaaataactttcaacagagaattcgcccggcgaataatacaatcagccttgagaataaatggggtaaactctcgttccagatccggacatcaggtgcaaatggaatgttttcggaggcagttgaattggaacgtgcaaatggcaaaaaatactatgtcaccgcagttgatcaagtaaaacccaaaatagcactcttgaagttcgtcgataaagatcctaaagggggaggcgggagcgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctg

SEQ ID NO: 12 - DNA, 45 bp, ARTIFICIAL SEQUENCE
  Primer
ggcggaagcggtaccgacgacgacgacaaggccgttcagggtcca

SEQ ID NO: 13 - DNA, 30 bp, ARTIFICIAL SEQUENCE
  Primer
gctcccgcctccccccagcagtttcaatgc

SEQ ID NO: 14 - DNA, 30 bp, ARTIFICIAL SEQUENCE
  Primer
gggggaggcgggagcggcctggacaccgtg

SEQ ID NO: 15 - DNA, 39 bp, ARTIFICIAL SEQUENCE
  Primer
gctcgtgtcgacctcgagtcattatttaggatctttatc

SEQ ID NO: 16 - DNA, 51 bp, ARTIFICIAL SEQUENCE
  Primer
agcccagatctgggtaccgacgacgacgacaagggcctggacaccgtgagc

SEQ ID NO: 17 - DNA, 27 bp, ARTIFICIAL SEQUENCE
  Primer
gctcccgcctccccctttaggatcttt

SEQ ID NO: 18 - DNA, 30 bp, ARTIFICIAL SEQUENCE
  Primer
gggggaggcgggagcgccgttcagggtcca

SEQ ID NO: 19 - DNA, 36 bp, ARTIFICIAL SEQUENCE
  Primer
gccgtcgacctcgagtcattacagcagtttcaatgc

SEQ ID NO: 20 - DNA, 1204 bp, HUMAN
gaaattcttacaaaaactgaaagtgaaatgaggaagacagattgagcaatccaatcggagggtaaatgccagcaaacctactgtacagtaggggtagagatgcagaaaggcagaaaggagaaaattcaggataactctcctgaggggtgagccaagccctgccatgtagtgcacgcaggacatcaacaaacacagataacaggaaatgatccattccctgtggtcacttattctaaaggccccaaccttcaaagttcaagtagtgatatggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtgacctacttacaccatgtctgtagctattttcctccctttctctgtacctctaagaagaaagaatctaactgaaaatacca

SEQ ID NO: 21 - protein, 316 aa, Plant
MKGNMKVYWIKIAVATWFCCTTIVLGSTARIFSLPTNDEEETSKTLGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKTSLAAELIIQNYESLVGFD

SEQ ID NO: 22 - DNA, 24 bp, Artificial Sequence
  Synthetic Primer
ggagaaggcaactccagtcagaac

SEQ ID NO: 23 - DNA, 24 bp, Artificial Sequence
  Synthetic Primer
gtccatgtctttggggatgaattg